SEQUENCE LISTING (54 sequences)

SEQ ID NO: 1 - protein, 454 aa, Saccharomyces cerevisiae
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 2 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLPGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 3 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRTNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVSLNVLSKCAGSKKFRPAPAAAFARECRGFYFELQELKEDDYYGITLSDDSDHQFLLANQVVVHN

SEQ ID NO: 4 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVEFVKEVSKSYPISEGPERANELVESYRKASNKAYFEWTIEARDLSPLGSHVRKATYQTYAPILYENDHFFDYMQKSKFHLTIEGPKVLAYLLGLWIGDGLSDRATFSVDSRDTSLMERVTEYAEKLNLCAEYKDRKEPRVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 5 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIGSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 6 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKAPNKAYLEWTIEARDLSLLGSHVRKATYQTYAPILYENDHFFDYMQKSKFHLTIEGPKVLAYLLGLWIGDGLSDRATFSVDSRDASLMERVTEYAEKLSLCAEYKDRKEPQVAKTVNLYSKVVRGNGIRNNLNTEDPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQAVVHN

SEQ ID NO: 7 - protein, 454 aa, Artificial Sequence
  Description of Artificial Sequence Synthetic VMA allele mutation
CFAKGTNVLMADGSIECIENIKVGNKVMGKDGRPREVIKLPRGRETVYSVVQKSQHRAHKSDSSREVPELLKFTCNATHELVVRTPRSVRRLSRTIKGVEYLEVITFEMGQKKAPDGRIVELVKEVSKSYPISEGPERANELVESYRKASNKAYFEWTIEARDLSLSGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 8 - protein, 454 aa, Artificial Sequence
  Description of Artificial Sequence Synthetic VMA allele mutation
CFAKGTNVLMADGSIECIENIEVGNKVMGKGGRPRGVIKLPRGRETMYSVVQKSQHRAHKSDPSREVPELLKFTCNATHELVVRTPRSVRRLSRTIKGVEYFEVITFEMGQKKAPDGRIVELVKEVSKSYPISEGPGRANELVESYRKASNKACFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLSTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 9 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFKVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARFLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 10 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFERTIEARDLSLLGSHVRKATYQTYAPILYENDHFFDYMQKSKFHLTIEGPKVLAYLLGLWIGDALSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 11 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVNVHN

SEQ ID NO: 12 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVTGHN

SEQ ID NO: 13 - DNA, 3096 bp, Saccharomyces cerevisiae
  CDS (1)..(3093)
atgattggttgtgccatgtacgaattggtcaaggtcggtcacgataacctggtgggtgaagtcattagaattgacggtgacaaggccaccatccaagtttacgaagaaactgcaggccttacggtcggtgaccctgttttgagaacaggtaagcctctgtcggtagaattgggtcctggtctgatggaaaccatttacgatggtattcaaagacctttgaaagccattaaggaagaatcgcaatcgatttatatcccaagaggtattgacactccagctttggataggactatcaagtggcaatttactccgggaaagtttcaagtcggcgatcatatttccggtggtgatatttacggttccgtttttgagaattcgctaatttcaagccataagattcttttgccaccaagatcaagaggtacaatcacttggattgctccagctggtgagtacactttggatgagaagattttggaagttgaatttgatggcaagaagtctgatttcactctttaccatacttggcctgttcgtgttccaagaccagttactgaaaagttatctgctgactatcctttgttaacaggtcaaagagttttggatgctttgtttccttgtgttcaaggtggtacgacatgtattccaggtgcttttggttgtggtaagaccgttatctctcaatctttgtccaagtactccaattctgacgccattatctatgtcgggtgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataattgcggagaaagaggtaatgaaatggcagaagtcttgatggaattcccagagttatatactgaaatgagcggtactaaagaaccaattatgaagcgtactactttggtcgctaatacatctaacatgccggttgcagccagagaagcttctatttacactggtatcactcttgcagaatacttcagagatcaaggtaaaaatgtttctatgattgcagactcttcttcaagatgggctgaagctttgagagaaatttctggtcgtttgggtgagatgcctgctgatcaaggtttcccagcttatttgggtgctaagttggcctccttttacgaaagagccggtaaagctgttgctttaggttccccagatcgtactggttccgtttccatcgttgctgccgtttcgccagccgatggtgatttctcagatcctgttactactgctacattgggtatcactcaagtcttttggggtttagacaagaaattggctcaaagaaagcatttcccatctatcaacacatctgtttcttactccaaatacactaatgtcttgaacaagttttatgattccaattaccctgaatttcctgttttaagagatcgtatgaaggaaattctatcaaacgctgaagaattagaacaagttgttcaattagttggtaaatcggccttgtctgatagtgataagattactttggatgttgccactttaatcaaggaagatttcttgcaacaaaatggttactccacttatgatgctttctgtccaatttggaagacatttgatatgatgagagccttcatctcgtatcatgacgaagctcaaaaagctgttgctaatggtgccaactggtcaaaactagctgactctactggtgacgttaagcatgccgtttcttcatctaaattttttgaaccaagcaggggtgaaaaggaagtccatggcgaattcgaaaaattgttgagcactatgcaagaaagatttgctgaatctaccgattaa

SEQ ID NO: 14 - protein, 1031 aa, Saccharomyces cerevisiae
MIGCAMYELVKVGHDNLVGEVIRIDGDKATIQVYEETAGLTVGDPVLRTGKPLSVELGPGLMETIYDGIQRPLKAIKEESQSIYIPRGIDTPALDRTIKWQFTPGKFQVGDHISGGDIYGSVFENSLISSHKILLPPRSRGTITWIAPAGEYTLDEKILEVEFDGKKSDFTLYHTWPVRVPRPVTEKLSADYPLLTGQRVLDALFPCVQGGTTCIPGAFGCGKTVISQSLSKYSNSDAIIYVGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHNCGERGNEMAEVLMEFPELYTEMSGTKEPIMKRTTLVANTSNMPVAAREASIYTGITLAEYFRDQGKNVSMIADSSSRWAEALREISGRLGEMPADQGFPAYLGAKLASFYERAGKAVALGSPDRTGSVSIVAAVSPADGDFSDPVTTATLGITQVFWGLDKKLAQRKHFPSINTSVSYSKYTNVLNKFYDSNYPEFPVLRDRMKEILSNAEELEQVVQLVGKSALSDSDKITLDVATLIKEDFLQQNGYSTYDAFCPIWKTFDMMRAFISYHDEAQKAVANGANWSKLADSTGDVKHAVSSSKFFEPSRGEKEVHGEFEKLLSTMQERFAESTD

SEQ ID NO: 15 - DNA, 3147 bp, Candida tropicalis
  CDS (1)..(3144)
atgattggatgtgccatgtacgaattggttaaagttggtcatgataatttagttggggaagttattagaattaatggtgataaagcaaccattcaagtttatgaagaaactgcaggggtcactgttggtgatccagttttaagaactggtaaaccattatctgttgaattaggtcctggtttaatggaaactatttatgatggtattcaaagacctttaaaagccattaaagatgaatcccaatctatttatatcccaagaggtattgatgttcctgctttatcaagaactgttcaatatgatttcactccaggtcaattgaaagttggtgatcatatcactggtggggacatttttggttctatttatgaaaactctttattggatgaccataagattttgttacctccaagagcaagaggtactattacttctattgctgaagccggttcttataatgttgaagaaccagttttggaagttgaatttgatggtaagaaacataaatactctatgatgcatacatggccagttagagttccaagaccagttgctgaaaaattgactgctgatcatccattgttgaccggtcaaagagtcttggattctttattcccatgtgttcaaggtggtactacttgtatcccaggggcttttggttgtggtaaaactgttatttctcaatctttgtccaaattctccaactctgatgttattatctatgttggttgtttcactaaaggtactcaagtcatgatggctgatggtgccgacaaatctattgaatctattgaagttggtgacaaagtcatgggtaaagatggtatgccaagagaagttgttggcttaccaagaggttatgatgatatgtacaaggttcgtcaactttctagtactagacgtaatgctaaatccgaaggcttgatggatttcactgtttctgctgatcataaacttatcttgaaaactaaacaagatgtcaagattgctacacgtaaaattggtggcaacacctatactggtgttactttctatgttttggaaaagactaagactggtattgaattagttaaagccaagactaaagttttcggtcatcatatccatggtcaaaatggcgctgaagaaaaagctgctacttttgctgctggcattgactctaaagaatacattgattggatcattgaagctagagattatgtacaagttgatgaaattgtcaagaccagcaccactcaaatgatcaacccagttcattttgaatctggtaaactcggtaactggttacacgaacacaagcaaaacaaatcacttgctccacaattgggttacttgttgggtacttgggctggtattggaaatgttaaatcttctgctttcaccatgaactccaaagatgatgttaaattagctacaagaattatgaactactcttcaaaattgggcatgacttgttcttctactgaatccggtgaactcaatgtcgctgaaaacgaagaagaatttttcaataaccttggtgctgaaaaggatgaagctggtgatttcacttttgatgaatttaccgatgctatggatgaattgactatcaatgttcatggtgcagctgcaagcaagaagaacaatttgttgtggaatgctttgaaatctcttggtttcagagccaagtctactgatattgtcaagagtattcctcaacatattgctgttgatgatattgttgtcagagaatctttgattgccggtttagttgatgctgctggtaatgttgaaaccaaatccaatggttctattgaagctgttgttagaacttctttcagacatgtcgctagaggtcttgtcaagattgctcattctttgggtattgaatcatctattaatattaaagatactcacattgatgctgctggtgttagacaagaatttgcttgtattgtcaatttgactggtgctccacttgctggtgttctttctaaatgtgcacttgcaagaaaccaaactccagttgtcaaatttaccagagacccagttttgttcaactttgatttgatcaaatctgcaaaagaaaactattatggtattactttggctgaagaaactgatcatcaattccttttatccaacatggccttggtgcacaactgtggtgaacgtggtaatgagatggctgaagttttgatggaattcccagaattgtttactgaaatttctggtagaaaagaaccaattatgaaacgtaccactttggttgccaatacttctaatatgccagtcgctgccagagaagcttctatttatactggtattacattggctgaatatttcagagatcaaggtaagaatgtttctatgattgctgattcttcttcacgttgggctgaagctttgagagaaatttctggtagattgggtgaaatgcctgctgatcaaggtttcccagcttatttgggtgctaaattggcttctttctatgagcgtgccggtaaagccactgctttgggttcaccagatagagttggttcagtttctattgttgctgctgtttctccagctggtggtgatttctctgatccagttactacttctactttgggtattactcaagttttctgggggttggataagaaattggcccaaagaaaacatttcccatctattaacaccagtgtttcttattctaaatacaccaatgttttgaacaaatactatgattccaactatccagaattcccacaattgagagacaaaattagagaaattttatctaatgctgaagaattggaacaagttgttcaattagttggtaaatctgcattgtctgattctgataagattactttagatgttgctaccttgattaaagaagatttcttgcaacaaaatggttattcttcatatgatgcattctgtccaatttggaagacttttgatatgatgagagcatttatttcatattatgatgaagcacaaaaagcaattgccaatggtgctcaatggtctaaattagctgaaagtactagtgatgttaaacatgctgtttcttcagctaaattctttgaaccatcaagaggtcaaaaagaaggtgaaaaagaatttggagatttattaaccactatctccgaaagatttgctgaagcttcagaataa

SEQ ID NO: 16 - protein, 1048 aa, Candida tropicalis
MIGCAMYELVKVGHDNLVGEVIRINGDKATIQVYEETAGVTVGDPVLRTGKPLSVELGPGLMETIYDGIQRPLKAIKDESQSIYIPRGIDVPALSRTVQYDFTPGQLKVGDHITGGDIFGSIYENSLLDDHKILLPPRARGTITSIAEAGSYNVEEPVLEVEFDGKKHKYSMMHTWPVRVPRPVAEKLTADHPLLTGQRVLDSLFPCVQGGTTCIPGAFGCGKTVISQSLSKFSNSDVIIYVGCFTKGTQVMMADGADKSIESIEVGDKVMGKDGMPREVVGLPRGYDDMYKVRQLSSTRRNAKSEGLMDFTVSADHKLILKTKQDVKIATRKIGGNTYTGVTFYVLEKTKTGIELVKAKTKVFGHHIHGQNGAEEKAATFAAGIDSKEYIDWIIEARDYVQVDEIVKTSTTQMINPVHFESGKLGNWLHEHKQNKSLAPQLGYLLGTWAGIGNVKSSAFTMNSKDDVKLATRIMNYSSKLGMTCSSTESGELNVAENEEEFFNNLGAEKDEAGDFTFDEFTDAMDELTINVHGAAASKKNNLLWNALKSLGFRAKSTDIVKSIPQHIAVDDIVVRESLIAGLVDAAGNVETKSNGSIEAVVRTSFRHVARGLVKIAHSLGIESSINIKDTHIDAAGVRQEFACIVNLTGAPLAGVLSKCALARNQTPVVKFTRDPVLFNFDLIKSAKENYYGITLAEETDHQFLLSNMALVHNCGERGNEMAEVLMEFPELFTEISGRKEPIMKRTTLVANTSNMPVAAREASIYTGITLAEYFRDQGKNVSMIADSSSRWAEALREISGRLGEMPADQGFPAYLGAKLASFYERAGKATALGSPDRVGSVSIVAAVSPAGGDFSDPVTTSTLGITQVFWGLDKKLAQRKHFPSINTSVSYSKYTNVLNKYYDSNYPEFPQLRDKIREILSNAEELEQVVQLVGKSALSDSDKITLDVATLIKEDFLQQNGYSSYDAFCPIWKTFDMMRAFISYYDEAQKAIANGAQWSKLAESTSDVKHAVSSAKFFEPSRGQKEGEKEFGDLLTTISERFAEASE

SEQ ID NO: 17 - DNA, 3033 bp, Chlamydomonas eugametos
  CDS (1)..(3030)
atgcctattggtgttccacgtattatttattgctggggagaagaacttcccgcacaatggactgatatttataactttatttttagacgtcgaatggtctttttaatgcaatatttggatgatgaactttgtaatcaaatctgtggtttattaattaatattcatatggaagaccgttcaaaagaattggaaaaaaaagaaattgaacgtagtggtttattcaaaggaggtccaaaaacacaaaaaggtgggacaggtgccggcgaaacaggtgcatcaagtattcaaaataaaaaaagcaatagttcatcatttgaagatttattagctgcagatgaggatttaggtattgatgaaaataatacattagaacaatatacacttcaaaaaattacaatggaatggttaaattggaatgctcaattttttgattattcagatgaaccttatcttttttatttagccgaaatgctatcaaaagattttaataaaggagatgctcgtatgttattttcaaataataataaattttcaatgccattttctcaaatgcttaatacaggatcgatgtccgatccacgtcgcccacagtctacgaacggggctaattggaattcaagtgaacaaaataattctttagacatttattctcctttccgtatgttagctaattttgaagcccaagattatgattttaaacaaattaatccatctttagcttcaaaagaagaagttttcaaactttttaataatactattttaaaaaatggaggtcaacgtaataataatatgtccaaattattaacagaattagcacaacgtaattgggaaaataaaacaaattcacaagaaaatttatataaaagcacagaaaaagctttgagtcaacgtaatttacgaaaagaatatattaaagaccgtactttaaataattattcaagtgacccgtttaatacaaaaggctacgtcaacgcacaaggtgcgtcgacggggccaagccctcgtacacgtggtatgcatgccgacggatccttaaattatttagatttctattcttataatgattcttataatgatttcaaaactgcacctcgtggaaaacaagctgaacgtgccttccaagaagaggaatctaaaaaagtttttgttattattaactcgtttggtggttctgttggtaatgggattactgtgcatgatgcacttcaatttattaaagctgggtcattaacattagctttaggtgttgcagcttccgccgcttcattagcccttgctggtggtactattggtgagcgttatgttacggaaggttgccatgttatgattcaccaaccagaatgcttgacttctgaccacactgtattaacaactcgcggttggattcctattgctgacgtaactcttgatgacaaagtagcggttttagataacaatacaggtgaaatgtcatatcaaaatccacaaaaagtacataaatatgactatgaaggtccaatgtatgaagtaaaaacagctggagttgacttatttgttacaccaaaccaccgtatgtatgttaacacaacgaataatactacgaaccaaaactataatttagttgaagcttcatctatttttggaaaaaaagtacgttacaaaaatgatgctatctggaataaaaccgattatcaatttattttacctgaaactgcaacgcttacaggtcatacaaataaaataagctctacacctgccatccaacccgaaatgaacgcttggctaactttctttggattatggatcgctaacggacatactacgaaaattgctgaaaaaacagcagaaaataatcaacaaaaacaacgatataaggtaattctgactcaagttaaagaagatgtttgtgatattattgaacaaactttaaataaattaggatttaattttattcgtagtggtaaagattacacaattgaaaataaacaactatggtcttacttaaatcctttcgataacggggctttaaataaatatttacctgattgggtatgggaattaagttcacaacaatgtaaaattttattaaatagcttatgtcttggtaattgtcttttcactaaaaacgatgacactttacattattttagtacgtcagaacgttttgcaaatgatgttagccgtttggccttacatgccggaacaacttcgactattcaattagaagcagctccaagtaatctatatgatacaattattggtctacctgttgaagtaaacactactctatggcgtgtaattattaatcaaagtagtttctactcttattccactgacaaatcaagcgcactaaatttatctaataatgtagcatgctacgtcaacgcgcagagcgcgttgacgttagaacaaaattctcaaaaaatcaataaaaatactttagttttaacaaaaaataacgtaaaaagtcaaacaatgcatagtcaacgcgcagagcgcgttgacacggctcttttaactcaaaaagagcttgataactcattaaatcatgaaattttaattaataaaaaccctggtactagtcaattagaatgtgtagttaaccctgaagttaataacacatcaactaatgatcgttttgtttactacaaagggccagtatattgcttaactggtcctaacaacgtattctacgtacaacgaaacggaaaagctgtgtggacaggtaacagttcaattcaaggccaagcatcagatatttggattgatagtcaagaaatcatgaaaattcgtttagatgtagcagaaatttattcattagctacttatcgtccgcgtcacaaaattttacgtgatttagatcgtgatttttatctaacggcaactgaaacaattcattatggtttagctgatgaaattgcttctaatgaagtaatgcaagaaattattgaaatgacaagtaaagtttgggactatcatgatacaaaacaacaacgtttactagaaagtcgtgattctacaacttctggggcagatacacaatctcaaaattaa

SEQ ID NO: 18 - protein, 1010 aa, Chlamydomonas eugametos
MPIGVPRIIYCWGEELPAQWTDIYNFIFRRRMVFLMQYLDDELCNQICGLLINIHMEDRSKELEKKEIERSGLFKGGPKTQKGGTGAGETGASSIQNKKSNSSSFEDLLAADEDLGIDENNTLEQYTLQKITMEWLNWNAQFFDYSDEPYLFYLAEMLSKDFNKGDARMLFSNNNKFSMPFSQMLNTGSMSDPRRPQSTNGANWNSSEQNNSLDIYSPFRMLANFEAQDYDFKQINPSLASKEEVFKLFNNTILKNGGQRNNNMSKLLTELAQRNWENKTNSQENLYKSTEKALSQRNLRKEYIKDRTLNNYSSDPFNTKGYVNAQGASTGPSPRTRGMHADGSLNYLDFYSYNDSYNDFKTAPRGKQAERAFQEEESKKVFVIINSFGGSVGNGITVHDALQFIKAGSLTLALGVAASAASLALAGGTIGERYVTEGCHVMIHQPECLTSDHTVLTTRGWIPIADVTLDDKVAVLDNNTGEMSYQNPQKVHKYDYEGPMYEVKTAGVDLFVTPNHRMYVNTTNNTTNQNYNLVEASSIFGKKVRYKNDAIWNKTDYQFILPETATLTGHTNKISSTPAIQPEMNAWLTFFGLWIANGHTTKIAEKTAENNQQKQRYKVILTQVKEDVCDIIEQTLNKLGFNFIRSGKDYTIENKQLWSYLNPFDNGALNKYLPDWVWELSSQQCKILLNSLCLGNCLFTKNDDTLHYFSTSERFANDVSRLALHAGTTSTIQLEAAPSNLYDTIIGLPVEVNTTLWRVIINQSSFYSYSTDKSSALNLSNNVACYVNAQSALTLEQNSQKINKNTLVLTKNNVKSQTMHSQRAERVDTALLTQKELDNSLNHEILINKNPGTSQLECVVNPEVNNTSTNDRFVYYKGPVYCLTGPNNVFYVQRNGKAVWTGNSSIQGQASDIWIDSQEIMKIRLDVAEIYSLATYRPRHKILRDLDRDFYLTATETIHYGLADEIASNEVMQEIIEMTSKVWDYHDTKQQRLLESRDSTTSGADTQSQN

SEQ ID NO: 19 - DNA, 2373 bp, Mycobacterium tuberculosis
  CDS (1)..(2370)
atgacgcagacccccgatcgggaaaaggcgctcgagctggcagtggcccagatcgagaagagttacggcaaaggttcggtgatgcgcctcggcgacgaggcgcgtcagccgatttcggtcattccgaccggatccatcgcactagacgtggccctgggcattggcggcctgccgcgtggccgggtgatagagatatacggcccggagtcgtcgggtaagaccaccgtggcgctgcacgcggtggccaacgctcaggccgccggtggtgttgcggcgttcatcgacgccgagcacgcgctggatccggactatgccaagaagctcggtgtcgacaccgattcgctgctggtcagccagccggacaccggggaacaggcactcgagatcgccgacatgctgatccgctcgggtgcgcttgacatcgtggtgatcgactcggtggcggcgctggtgccgcgcgcggagctcgaaggcgagatgggcgacagccacgtcgggctgcaggcccggctgatgagccaggcgctgcggaaaatgaccggcgcgctgaataattcgggcaccacggcgatcttcatcaaccagctccgcgacaagatcggagtgatgttcgggtcgcccgagacgacaacgggcggaaaggcgttgaagttctacgcgtcggtgcgcatggacgtgcggcgagtcgagacgctcaaggacggtaccaacgcggtcggcaaccgcacccgggtcaaggtcgtcaagaacaagtgcctcgcagagggcactcggatcttcgatccggtcaccggtacaacgcatcgcatcgaggatgttgtcgatgggcgcaagcctattcatgtcgtggctgctgccaaggacggaacgctgcatgcgcggcccgtggtgtcctggttcgaccagggaacgcgggatgtgatcgggttgcggatcgccggtggcgccatcgtgtgggcgacacccgatcacaaggtgctgacagagtacggctggcgtgccgccggggaactccgcaagggagacagggtggcgcaaccgcgacgcttcgatggattcggtgacagtgcgccgattccggcggatcatgcccggctgcttggctacctgatcggagatggcagggatggttgggtggggggcaagactccgatcaacttcatcaatgttcagcgggcgctcattgacgacgtgacgcgaatcgctgcgacgctcggttgcgcggcccatccgcaggggcgtatctcactcgcgatcgctcatcgacccggtgagcgcaacggtgtggcagacctttgtcagcaggccggtatctacggcaagctcgcgtgggagaagacgattccgaattggttcttcgagccggacatcgcggccgacattgtcggcaatctgctcttcggcctgttcgaaagcgacgggtgggtgagccgggaacagaccggggcacttcgggtcggttacacgacgacctctgaacaactcgcgcatcagattcattggctgctgctgcggttcggtgtcgggagcaccgttcgagattacgatccgacccagaagcggccgagcatcgtcaacggtcgacggatccagagcaaacgtcaagtgttcgaggtccggatctcgggtatggataacgtcacggcattcgcggagtcagttcccatgtgggggccgcgcggtgccgcgcttatccaggcgattccagaagccacgcaggggcggcgtcgtggatcgcaagcgacatatctggctgcagagatgaccgatgccgtgctgaattatctggacgagcgcggcgtgaccgcgcaggaggccgcggccatgatcggtgtagcttccggggacccccgcggtggaatgaagcaggtcttaggtgccagccgccttcgtcgggatcgcgtgcaggcgctcgcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgtcgtgcacaactgttcgccccccttcaagcaggccgagttcgacatcctctacggcaagggaatcagcagggagggctcgctgatcgacatgggtgtggatcagggcctcatccgcaagtcgggtgcctggttcacctacgagggcgagcagctcggccagggcaaggagaatgcccgcaacttcttggtggagaacgccgacgtggctgacgagatcgagaagaagatcaaggaaaagcttggcattggtgccgtggtgaccgatgatccctcaaatgacggtgtcctgcccgcccccgtcgacttctga

SEQ ID NO: 20 - protein, 790 aa, Mycobacterium tuberculosis
MTQTPDREKALELAVAQIEKSYGKGSVMRLGDEARQPISVIPTGSIALDVALGIGGLPRGRVIEIYGPESSGKTTVALHAVANAQAAGGVAAFIDAEHALDPDYAKKLGVDTDSLLVSQPDTGEQALEIADMLIRSGALDIVVIDSVAALVPRAELEGEMGDSHVGLQARLMSQALRKMTGALNNSGTTAIFINQLRDKIGVMFGSPETTTGGKALKFYASVRMDVRRVETLKDGTNAVGNRTRVKVVKNKCLAEGTRIFDPVTGTTHRIEDVVDGRKPIHVVAAAKDGTLHARPVVSWFDQGTRDVIGLRIAGGAIVWATPDHKVLTEYGWRAAGELRKGDRVAQPRRFDGFGDSAPIPADHARLLGYLIGDGRDGWVGGKTPINFINVQRALIDDVTRIAATLGCAAHPQGRISLAIAHRPGERNGVADLCQQAGIYGKLAWEKTIPNWFFEPDIAADIVGNLLFGLFESDGWVSREQTGALRVGYTTTSEQLAHQIHWLLLRFGVGSTVRDYDPTQKRPSIVNGRRIQSKRQVFEVRISGMDNVTAFAESVPMWGPRGAALIQAIPEATQGRRRGSQATYLAAEMTDAVLNYLDERGVTAQEAAAMIGVASGDPRGGMKQVLGASRLRRDRVQALADALDDKFLHDMLAEELRYSVIREVLPTRRARTFDLEVEELHTLVAEGVVVHNCSPPFKQAEFDILYGKGISREGSLIDMGVDQGLIRKSGAWFTYEGEQLGQGKENARNFLVENADVADEIEKKIKEKLGIGAVVTDDPSNDGVLPAPVDF

SEQ ID NO: 21 - DNA, 4047 bp, Saccharomyces cerevisiae
  CDS (6)..(4037)
gaaagatgaagctactgtcttctatcgaacaagcatgcgatatttgccgacttaaaaagcttaaatgctttgccaagggaacgaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcctattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataattgctccaaagaaaaaccgaagtgcgccaagtgtcttaagaacaactgggagtgtcgctactctcccaaaaccaaaaggtctccgctgactagagctcatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattgactcggcagctcatcatgataactccacaattccgttggattttatgcccagggatgctcttcatggatttgattggtctgaagaggatgacatgtcggatggcttgcccttcctgaaaacggaccccaacaataatgggttctttggcgacggttctctcttatgtattcttcgatctattggctttaaaccggaaaattacacgaactctaacgttaacaggctcccgaccatgattacggatagatacacgttggcttctagatccacaacatcccgtttacttcaaagttatctcaataattttcacccctactgccctatcgtgcactcaccgacgctaatgatgttgtataataaccagattgaaatcgcgtcgaaggatcaatggcaaatcctttttaactgcatattagccattggagcctggtgtatagagggggaatctactgatatagatgttttttactatcaaaatgctaaatctcatttgacgagcaaggtcttcgagtcaggttccataattttggtgacagccctacatcttctgtcgcgatatacacagtggaggcagaaaacaaatactagctataattttcacagcttttccataagaatggccatatcattgggcttgaatagggacctcccctcgtccttcagtgatagcagcattctggaacaaagacgccgaatttggtggtctgtctactcttgggagatccaattgtccctgctttatggtcgatccatccagctttctcagaatacaatctccttcccttcttctgtcgacgatgtgcagcgtaccacaacaggtcccaccatatatcatggcatcattgaaacagcaaggctcttacaagttttcacaaaaatctatgaactagacaaaacagtaactgcagaaaaaagtcctatatgtgcaaaaaaatgcttgatgatttgtaatgagattgaggaggtttcgagacaggcaccaaagtttttacaaatggatatttccaccaccgctctaaccaatttgttgaaggaacacccttggctatcctttacaagattcgaactgaagtggaaacagttgtctcttatcatttatgtattaagagattttttcactaattttacccagaaaaagtcacaactagaacaggatcaaaatgatcatcaaagttatgaagttaaacgatgctccatcatgttaagcgatgcagcacaaagaactgttatgtctgtaagtagctatatggacaatcataatgtcaccccatattttgcctggaattgttcttattacttgttcaatgcagtcctagtacccataaagactctactctcaaactcaaaatcgaatgctgagaataacgagaccgcacaattattacaacaaattaacactgttctgatgctattaaaaaaactggccacttttaaaatccagacttgtgaaaaatacattcaagtactggaagaggtatgtgcgccgtttctgttatcacagtgtgcaatcccattaccgcatatcagttataacaatagtaatggtagcgccattaaaaatattgtcggttctgcaactatcgcccaataccctactcttccggaggaaaatgtcaacaatatcagtgttaaatatgtttctcctggctcagtagggccttcacctgtgccattgaaatcaggagcaagtttcagtgatctagtcaagctgttatctaaccgtccaccctctcgtaactctccagtgacaataccaagaagcacaccttcgcatcgctcagtcacgccttttctagggcaacagcaacagctgcaatcattagtgccactgaccccgtctgctttgtttggtggcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagcagaagctgatctccgaggaggatctgtaggtacccc

SEQ ID NO: 22 - protein, 1344 aa, Saccharomyces cerevisiae
MKLLSSIEQACDICRLKKLKCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHNCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEQKLISEEDL

SEQ ID NO: 23 - protein, 13 aa, Artificial Sequence
  Description of Artificial Sequence Conserved N1 domain
CFAKGTNVLMADG

SEQ ID NO: 24 - protein, 7 aa, Artificial Sequence
  Description of Artificial Sequence Conserved N2 domain
IEVGNKV

SEQ ID NO: 25 - protein, 14 aa, Artificial Sequence
  Description of Artificial Sequence Conserved N3 domain
LLKFTCNATHELVV

SEQ ID NO: 26 - protein, 16 aa, Artificial Sequence
  Description of Artificial Sequence Conserved N4 domain
WKLIDEIKPGDYAVLQ

SEQ ID NO: 27 - protein, 9 aa, Artificial Sequence
  Description of Artificial Sequence Conserved EN1 domain
LLGLWIGDG

SEQ ID NO: 28 - protein, 8 aa, Artificial Sequence
  Description of Artificial Sequence Conserved EN2 domain
VKNIPSFL

SEQ ID NO: 29 - protein, 10 aa, Artificial Sequence
  Description of Artificial Sequence Conserved EN3 domain
FLAGLIDSDG

SEQ ID NO: 30 - protein, 19 aa, Artificial Sequence
  Description of Artificial Sequence Conserved EN4 domain
TIHTSVRDGLVSLARSLGL

SEQ ID NO: 31 - protein, 8 aa, Artificial Sequence
  Description of Artificial Sequence Conserved C1 domain
NQVVVHNC

SEQ ID NO: 32 - protein, 14 aa, Artificial Sequence
  Description of Artificial Sequence Conserved C2 domain
YGITLSDDSDHQFL

SEQ ID NO: 33 - protein, 13 aa, Artificial Sequence
  Description of Artificial Sequence Conserved N1 domain
CXXXDXXXXXXXG

SEQ ID NO: 34 - protein, 7 aa, Artificial Sequence
  Description of Artificial Sequence Conserved N2 domain
XXXGXXV

SEQ ID NO: 35 - protein, 14 aa, Artificial Sequence
  Description of Artificial Sequence Conserved N3 domain
GXXXXXTXXHXXXX

SEQ ID NO: 36 - protein, 16 aa, Artificial Sequence
  Description of Artificial Sequence Conserved N4 domain
WXXXXXXXXXDXXXXX

SEQ ID NO: 37 - protein, 9 aa, Artificial Sequence
  Description of Artificial Sequence Conserved EN1 domain
LXGXXXXXG

SEQ ID NO: 38 - protein, 8 aa, Artificial Sequence
  Description of Artificial Sequence Conserved EN2 domain
XKXIPXXX

SEQ ID NO: 39 - protein, 10 aa, Artificial Sequence
  Description of Artificial Sequence Conserved EN3 domain
XLXGXFXXDG

SEQ ID NO: 40 - protein, 19 aa, Artificial Sequence
  Description of Artificial Sequence Conserved EN4 domain
XXSXXXXXXXXXLLXXXGI

SEQ ID NO: 41 - protein, 14 aa, Artificial Sequence
  Description of Artificial Sequence Conserved C1 domain
XVYDLXVXXXXXFX

SEQ ID NO: 42 - protein, 8 aa, Artificial Sequence
  Description of Artificial Sequence Conserved C2 domain
NGXXXHNX

SEQ ID NO: 43 - protein, 454 aa, Artificial Sequence
  Description of Artificial Sequence Synthetic VMA allele mutation
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVKAHN

SEQ ID NO: 44 - protein, 15 aa, Artificial Sequence
  Description of Artificial Sequence Synthetic linker
GGGGSGGGGSGGGGS

SEQ ID NO: 45 - DNA, 12 bp, Artificial Sequence
  Description of Artificial Sequence Synthetic nucleic acid
aaaaagcttaag

SEQ ID NO: 46 - DNA, 35 bp, Artificial Sequence
  Description of Artificial Sequence Primer
tccaaagaaaaaccgaagtgcccaagtgtcttaag

SEQ ID NO: 47 - protein, 277 aa, Herpes simplex virus type 2
LLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLALFRVPELLAPNGDLYHIFAWVLDVLADRLLPMHLF

SEQ ID NO: 48 - protein, 269 aa, Bovine herpesvirus 1
LLRVYVDGPHGLGKTTAASRLASERGDAIYLPEPMSYWSGAGEDDLVARVYTAQHRMDRGEIDAREAAGVVLGAQLTMSTPYVALNGLIAPHIGEEPSPGNATPPDLILIFDRHPTASLLCYPLARYLTRCLPIESVLSLIALIPPTPPGTNLILGTAPAEDHLSRLVARGPPGELPDARMLRAIRYVYALLANTVKYLQSGGSWRADLGSEPPRLPLAPPEIGDPNNPGGHNTLLALIHGAGATRGCAAMTSWTLDLLADRLRSMNMF

SEQ ID NO: 49 - protein, 325 aa, Herpes simplex virus type 2
LLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLALFRVPELLAPNGDLYHIFAWVLDVLADRLLPMHLFVLDYDQSPVGCRDALLRLTAGMIPTRVTTAGSIAEIRDLARTFAREVG

SEQ ID NO: 50 - protein, 317 aa, Pseudorabies virus
ILRIYLDGAYDTGKSTTARVMALGGALYVPEPMAYWRTLFDTDTVAGIYDAQTRKQNGSLSEEDAALVTAHDQAAFATPYLLLHTRLVPLFGPAVEGPPEMTVVFDRHPVAATVCFPLARFIVGDISAAAFVGLAATLPGEPPGGNLVVASLDPDEHLRRLRARARAGEHVDARLLTALRNVYAMLVNTSRYLSSGRRWRDDWGRAPRFDQTTRDCLALNELCRPRDDPELQDTLFGAYKAPELCDRRGRPLEVHAWAMDALVAKLLPLRVSTVDLGPSPRVCAAAVAAQTRGMEVTESAYGDHIRQCVCAFTSEMG

SEQ ID NO: 51 - protein, 64 aa, Artificial Sequence
  Description of Artificial Sequence Illustrative peptide
FFLLSSSSYYXXCCXWLLLLPPPPHHQQRRRRIIIMTTTTNNKKSSRRVVVVAAAADDEEGGGG

SEQ ID NO: 52 - DNA, 64 bp, Artificial Sequence
  Description of Artificial Sequence Illustrative nucleic acid
ttttttttttttttttccccccccccccccccaaaaaaaaaaaaaaaagggggggggggggggg

SEQ ID NO: 53 - DNA, 64 bp, Artificial Sequence
  Description of Artificial Sequence Illustrative nucleic acid
ttttccccaaaaggggttttccccaaaaggggttttccccaaaaggggttttccccaaaagggg

SEQ ID NO: 54 - DNA, 64 bp, Artificial Sequence
  Description of Artificial Sequence Illustrative nucleic acid
tcagtcagtcagtcagtcagtcagtcagtcagtcagtcagtcagtcagtcagtcagtcagtcag

(19) United States
(12) Patent Application Publication
(10) Pub. No.: US 2004/0091966 A1
(43) Pub. Date: May 13, 2004

(54) Polypeptide regulation by conditional inteins

(76) Inventors: Martin Zeidler, Boston, MA (US); Norbert Perrimon, Arlington, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/441,147

(22) Filed: May 19, 2003

Related U.S. Application Data
(63) Continuation of application No. 09/651,768, filed on Aug. 30, 2000, now abandoned.
(60) Provisional application No. 60/151,600, filed on Aug. 30, 1999.

(51) Int. Cl. C12P 21/02; C07H 21/04; C12P 21/06; C07K 14/47; C12N 15/85

(52) U.S. Cl. 435/69.1; 435/455; 435/320.1; 435/325; 530/350; 536/23.5

(57) ABSTRACT

The present invention relates to methods and reagents for the regulation of a target polypeptide bioactivity by controlled self-excision of an intein.

### 1. BACKGROUND OF THE INVENTION

The polypeptide products of genes carry a wide assortment of bioactivities which effect most of the processes required for life including enzymatic functions, structural functions and the vast majority of biological control functions. Manipulation of these functions for experimental, agricultural or pharmaceutical purposes generally requires polypeptide-specific agonists or antagonists which, respectively, increase or decrease the particular bioactivity of interest. The rational design of small molecule agonist and antagonist ligands is advancing with new strides in the ability to predict target protein structure as well as with advances in combinatorial chemical synthesis and high through-put screening methodology. Nevertheless, a generally applicable method for controlling the biological activity of a preexisting polypeptide would obviate the need to identify novel and specific polypeptide agonists and antagonists as new biologically important target proteins are uncovered. Furthermore, potential unintended side-effects of a novel polypeptide agonist or antagonist would be prevented with a general method which is responsive to a known biological signal with predictable effects. Conditional mutations provide a means of regulating a particular target polypeptide in response to a particular regulatory signal. For example, temperature-sensitive conditional mutants are responsive to changes in temperature and generally evince reduced bioactivity at a particular temperature, the nonpermissive temperature, which is higher than that of the permissive temperature, at which bioactivity is greater. In contrast cold-sensitive mutants generally evince reduced bioactivity at a nonpermissive temperature which is lower than that of the permissive temperature. The use of such “conditional” mutants is particularly advantageous when studying the function of polypeptides which are “essential” for life—i.e. those polypeptides which encode a bioactivity which is essential for cell survival. Temperature sensitive mutations in a gene are generally isolated by means of extensive genetic screening for particular missense mutations in the target gene which render the encoded polypeptide thermolabile.

The heat-inducible N-degron module (U.S. Pat. No. 5,705,387) is a polypeptide structure which, when genetically engineered onto the amino-terminus of a target polypeptide, renders the target polypeptide thermolabile via a mechanism which involves N-end rule dependent proteolysis. Notably, this system results in the rapid degradation of the target polypeptide in the repressed state and so reactivation of the target requires new protein synthesis.

### 2. SUMMARY OF THE INVENTION

The present invention contemplates a general method for controlling a target polypeptide bioactivity by engineering the target protein with an inactivating polypeptide insert which can be regulatably excised from the target protein to yield native, biologically active protein in a controlled manner. In preferred embodiments of the invention, the inactivating polypeptide insert employed is a regulatable intein which is introduced into the host protein by genetic engineering of the host polypetide encoding gene. Inteins are protein-splicing elements that exist as in-frame fusions with flanking protein sequences called exteins. Naturally occurring inteins are appear to constitutively self-splice at the protein level, with their excision being coupled to extein ligation (see e.g. Cooper et al. (1995) TIBS 20: 351-56). At least some inteins encode an endonuclease activity which, once the intein has auto-excised from the host protein, can act to mediate the movement of the insertional element to new sites in the host organism's genome (Cooper et al. (1993) BioEssays 15: 667-73). Inteins are phylogenetically widespread, occurring in all three biological kingdoms—eubacteria, archaebacteria and eukaryotes. The terms extein and intein, as used herein, refer to both the genetic material and corresponding protein products.

The self-splicing mechanism of inteins has been well characterized and is known to one of ordinary skill in the art. The Intein Database at http://www.neb.com/ neb/inteins/html sets forth the general mechanism in detail. Without wishing to be bound to any theory, we set forth the mechanism as known in the art. In general, protein splicing involves four nucleophilic displacements by the 3 conserved splice junction residues. The conserved histidine residue present in the C1 block of the intein assists in Asparagine cyclization and C-terminal cleavage (Xu et al. (1996) EMBO 15(19):5146-5153) by hydrogen bonding to the Asparagine carbonyl oxygen, making this peptide bond more labile. The Threonine and Histidine in conserved block N3 assist in the initial acyl rearrangement at the N-terminal splice junction by hydrogen bonding to main chain atoms and holding the residue preceding the intein in a non-standard cis conformation. Any residue that can form similar hydrogen bonds can substitute for these conserved facilitating residues in Blocks N3 and C1. The mechanism of protein splicing has recently been reviewed by Perler et al. (1997) Nuc. Acids Res. 25:1087-93 and Shao et al. (1997) Chem. & Biol. 4:187-194. Since this mechanism is well documented in the art designing inteins which retain the self-splicing activity is considered to be well within the purview of the skilled artisan.

Regulation of the “target polypeptides” on-demand by the method of the present invention is achieved by introducing regulatable protein introns or inteins into the target polypeptides by methods known to the skilled artisan such as homologous recombination. Inteins are a group of related protein elements that are found within a range of host proteins immediately after their translation. Proteins containing the embedded inteins are non-functional. After translation the intein auto-catalytically splices itself out resulting in a functional host protein and an autonomous intein. Regulation of the self-splicing mechanism so that the self-splicing occurs on demand results in a process which will provide the host or target protein “on-demand”.

In particular, the self-splicing activity may be agonized or antagonized in response to a signal. Such signals include but are not limited to various internal and external factors including an increase or decrease in temperature, pH, exposure to light, unblocking of amino acid residues by dephosphorylation or deglycosylation, ionic concentrations, concentration of various metals, osmolarity, and/or the presence or absence of certain exogenous chemical agents such as various chemical dimerizer agents inducing rapamycin and related agents such as AP1510. Examples of exogenous chemicals include agents such as rapamycin or rapamycin analogs useful in mammalian systems and chemicals such as salicylic acid, abscissic acid useful in plant systems. Regulation of self-splicing of an engineered polypeptide at will via a regulating intermediate that could be easily supplied exogenously is particularly advantageous. This allows the production of the functional polypeptide as a function of the exogenously supplied chemical compound.

This allows control of the formation of the functional target polypeptide so that it is formed only at the appropriate time and to the appropriate extent, and in some situations in particular parts of the living system. In view of considerations like these, as well as others, it is clear that control of the time, extent and/or site of expression of the chimeric gene in plants or plant tissues would be highly desirable. Control that could be exercised easily would be of particular commercial value.

Other features and advantages of the invention will be apparent from the following detailed description and claims.

### 3. BRIEF DESCRIPTION OF THE FIGURES

FIG. 1 shows an intein splicing mechanism.

FIG. 2 shows the genetic modification of a generalized target gene with a regulatable intein, resulting in regulation of the encoded polypeptide bioactivity by controlled intein excision.

FIG. 3 shows the regulation of a polypeptide bioactivity by means of controlled intein trans-splicing with an organic dimerizer drug.

FIG. 4 shows the amino acid sequence of the yeast Sce intein and the positions location of allelic changes in conditional mutants. Conserved intein sequence motifs are underlined and numbering is relative to the first amino acid of the intein sequence. The positions of amino acid changes resulting in conditional temperature sensitive (TS) or cold sensitive (CS) mutations are shown as subscripts and the precise amino acid changes are indicated below the sequence where the first letter indicates the single letter designation of the intein amino occurring at the amino acid position designated by the number and the second letter indicates the identity of the substituted amino acid in the mutant. Conditional mutants associated with a single amino acid change are indicated as upper case TS and CS alleles while those associated with more than one alteration are indicated as lower case ts and cs alleles.

FIG. 5 shows the nucleic acid and amino acid sequence of the Saccharomyces cerevisiae VMA intein-containing TFP1-480 gene (GenBank Accession No. M21609). Numbering of the nucleotide sequence is in accordance with the GenBank entry and the intein-encoding nucleic acid sequence is underlined.

FIG. 6 shows the nucleic acid and amino acid sequence of the Candida tropicalis VMA intein-containing gene (GenBank Accession No. M64984). Numbering of the nucleotide sequence is in accordance with the GenBank entry and the intein-encoding nucleic acid sequence is underlined.

FIG. 7 shows the nucleic acid and amino acid sequence of the Chlamydomonas eugamentos clpP intein-containing gene (GenBank Accession No. L29402). Numbering of the nucleotide sequence is in accordance with the GenBank entry and the intein-encoding nucleic acid sequence is underlined.

FIG. 8 shows the nucleic acid and amino acid sequence of the Mycobacterium tuberculosis recA intein-containing gene (GenBank Accession No. X58485). Numbering of the nucleotide sequence is in accordance with the GenBank entry and the intein-encoding nucleic acid sequence is underlined.

FIG. 9 shows the nucleic acid and amino acid sequence of the GAL4::Sce VAM intein construct used to obtain conditional intein excision alleles.

FIG. 10 shows a Western blot analysis of the conditional Gal4:INT hydrid constructs.

### 4. DETAILED DESCRIPTION OF THE INVENTION

### 4.1. General

The invention provides compositions and methods for increasing or decreasing the bioactivity of a protein of interest, i.e., a regulatable target protein, by regulating the excision of a protein intron or intein inserted into the target polypeptide. In a preferred embodiment, the bioactivity of the target protein is regulated by inserting an intein encoding intein excision activity into the target protein, such that, the excision activity of the intein may be agonized or antagonized in response to a signal. The preferred signals include, but are not limited to, an increase or decrease in temperature, pH, exposure to light, unblocking of amino acid residues by dephosphorylation or deglycosylation, ionic concentrations, concentration of various metals, osmolarity, and/or the presence or absence of certain exogenous chemical agents or ligands.

The present invention is also directed to compositions comprising the modified target proteins and methods of their production. The modified proteins comprise a regulatable intein sequence inserted into the target protein, wherein the intein is capable of self-excision from the modified protein under predetermined conditions, i.e., an increase or decrease in temperature, pH, exposure to light, unblocking of amino acid residues by dephosphorylation or deglycosylation, ionic concentrations, concentration of various metals, osmolarity, and/or the presence or absence of certain exogenous chemical agents or ligands. If desired, the intein can be inserted into a region of the target proetin such that the bioactivity of the target protein is substantially inactivated. Accordingly, the bioactivity of the target polypeptide may be turned “on” or “off” on demand.

Other aspects of the invention are described below or will be apparent to those skilled in the art in light of the present disclosure.

### 4.2. Definitions

For convenience, the meaning of certain terms and phrases employed in the specification, examples, and appended claims are provided below.

As used herein, the terms “biological activity,” “bioactivity,” “activity” or “biological function” of a polypeptide or target polypeptide, are used interchangeably and refer to the catalytic, signaling, structural or other biological function of the given polypeptide. Biological activities include, for example, binding to a target peptide, e.g., the binding of a hormone receptor to a hormone. As used herein the term “bioactivity” may correspond to any catalytic activity of a polypeptide such as a kinase activity, a ligase activity, a phosphatase activity, a protease activity, or a polymerase activity. Subject “bioactivities” further include polypeptide sequences which function as protein, nucleic acid, lipid or small molecule recognition domains such as an antigenic determinant, a phosphorylation site, a DNA binding domain, an RNA binding domain, a secretion signal, a nuclear localization signal, a glycosylation site, a myristilation site, a homodimerization or heterodimerization domain or other protein interaction domain such as can be identified by the skilled artisan using two-hybrid interaction screening or polypeptide display panning methodologies.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “chimeric polypeptide” refers generally to a polypeptide comprising two subunits which do not occur together in the same polypeptide in nature, or at least, if present within the same polypeptide in nature, wherein the subunits do not occur in the same order in nature as in the chimeric polypeptide. When referring to the chimeric polypeptide of the invention, the term refers to a polypeptide comprising at least two functional subunits, a first functional subunit comprising portions of a target protein, and a second functional subunit which comprises a protein intron or intein. The terms “chimeric polypeptide” or “fusion polypeptide” or “hybrid polypeptide,” as used herein interchangeably, refer to a covalent joining of a first amino acid sequence encoding an intein polypeptide with a second amino acid sequence defining a target polypeptide. In general, an intein fusion polypeptide can be represented by the general formula N-INT-C, wherein INT represents a wild-type intein with constitutive autoexcision activity or a conditional intein derivative with inducible autoexcision activity and N and C refer to amino- and carboxy-terminal fragments of the target polypeptide respectively. In trans-spliced embodiments of the invention, two hydrid polypeptides which can be represented by the general formulae N-INT^{N} and INT^{C}-C, wherein INT^{N} comprises an amino-terminal fragment of an intein and INT^{C} comprises a carboxy-terminal fragment of an intein.

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids shown in, for example, SEQ ID No. 1 due to the degeneracy of the genetic code. “Equivalent polypeptides” of the invention are understood to include polypeptides related to those disclosed by one or more amino acid substitutions corresponding to conservative changes (i.e. those changes observed frequently within evolutionarily divergent homologs). The “equivalent polypeptides” of the invention further include equivalent conditional intein polypeptides, such as those obtained by altering any known intein polypeptide sequence so as to correspond to the mutant conditional intein sequences disclosed herein.

The term “extein” refers to a segment of a target polypeptide which is joined to an intein sequence. An N-extein is an amino-terminal portion of a target polypeptide which is joined at its carboxy-terminal end to an intein polypeptide. A C-extein is a carboxy-terminal portion of the target polypeptide which is joined at its amino-terminal end to an intein polypeptide. As used herein, the term “extein” is used in reference to both nucleic acid sequences which encode the amino-terminal and carboxy-terminal portion of the target polypeptides as well as the encoded target polypeptide segments themselves. Typically, subject exteins of the invention are produced as chimeric polypeptides having the general formula N-Extein/Intein/C-Extein. The term “heterologous” or expressions “heterologous protein” or “heterologous target,” as used herein, refer to any polypeptide sequence encoding a bioactivity to be regulated by a subject regulatable intein, and which polypeptide sequence does not occur in nature as an intein chimeric protein of the particular structure or sequence to be used in the method of the present invention. Thus subject heterologous proteins generally encode any “bioactivity” to be regulated by a regulatable intein. Preferred heterologous targets are mammalian proteins, particularly human proteins.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the target protein sequences of the present invention.

As used herein the terms “percent homology” or “percent identity” refer to degrees of similarity between two or more nucleic acids or two or more polypeptides which are defined by various mathematical algorithms which have been developed in the art. For example, percent identity can be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

“Inteins” or “protein introns” of this invention include intron-like elements that are removed post-translationally from the target protein in which they are embedded in-frame, by self-splicing. In other words, inteins are splicing elements that occur naturally as in-frame protein fusions, these inteins are not removed from RNA transcripts, but are translated in-frame as part the target protein in which they are inserted. Self-excision of the intein is followed by ligation of the two external remaining sequences of the target protein to produce an active functional protein. The external target sequences are called exteins. The term intein, as used herein includes within its scope naturally occurring isolated and/or purified intein polypeptides, fragments comprising intein elements minimally required for self-splicing, for example inteins comprising the N- and C-terminal domains of the inteins linked with a linker moiety, trans-spliced inteins, synthetically designed inteins, condition-sensitive mutants. The term includes both naturally occurring inteins as well as recombinant or synthetic inteins. As used herein, the term intein includes the nucleic acids encoding the autonomous polypeptides and the polypeptide itself.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature. An interaction can be direct or indirect, i.e., mediated by another molecule. Two molecules interacting directly are also referred to as binding to each other.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject intein polypeptides preferably includes no more than 10 kilobases (kb) of nucleic acid sequence which naturally immediately flanks the intein coding sequence DNA, more preferably no more than 5 kb of such naturally occurring cDNA or genomic flanking sequences, and most preferably less than 1.5 kb of such flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin. In preferred embodiments, a regulatable intein is inserted or “knocked-into” a target gene of the transgenic animal so as to render one or more bioactivities encoded by the target gene polypeptide subject to regulation by controlled intein excision.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.). In preferred embodimbents, the “knock-out” gene locus corresponding to the modified endogenous gene no longer encodes a functional polypeptide activity and is said to be a “null” allele. Accordingly, knock-out transgenic animals of the present invention include those carrying one target gene null mutation, i.e. a target gene null allele heterozygous animals, and those carrying two target gene null mutations, such as a target gene null allele homozygous animals.

A “knock-out construct” refers to a nucleic acid sequence that can be used to decrease or suppress expression of a protein encoded by endogenous DNA sequences in a cell. In a simple example, the knock-out construct is comprised of a hypothetical target gene with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: TARGET 5′/neo/TARGET 3′, where TARGET 5′ and TARGET 3′, refer to genomic or cDNA sequences which are, respectively, upstream and downstream relative to a portion of the TARGET gene and where neo refers to a neomycin resistance gene. In another knock-out construct, a second selectable marker is added in a flanking position so that the gene can be represented as: TARGET/neo/TARGET/TK, where TK is a thymidine kinase gene which can be added to either the TARGET 5′ or the TARGET 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)) of an activity and, preferably, a polypeptide bioactivity.

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant genes, e.g., gene encoding a chimeric polypeptide, is present and/or expressed or disrupted in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a sequence shown in one of the sequence listings. Nucleic acids at least 90%, more preferably 95%, and most preferably at least about 98-99% identical with a nucleic sequence represented in one of the sequence listings are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48: 443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

A “polymorphic gene” refers to a gene having at least one polymorphic region.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product. The term polypeptide includes peptidomimetics.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous protein. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations including substitutions and deletions (including truncation) of a naturally occurring form of the polypeptide.

The term “regulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “signal” as used refers to any chemical, physical or energetic agent which can be used to alter the autoexcision activity of the subject regulatable inteins. Examples include of signals contemplated in the instant invention include: temperature changes (either increases or decreases in temperature); pH changes; changes in salt concentration; changes in ionic strength; exposure to electromagnetic radiation; and changes in pressure. Subject signals of the invention further include chemical signals such as signals produced by the addition or removal of: a chemical ligand (preferably a bivalent dimerizing agent); a metal ion; a carbohydrate moiety; a lipid moiety; a nucleic acid; or a polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention, e.g., to identify compounds that modulate the interaction between two polypeptides.

As used herein, the term “specifically hybridizes” or “specifically detects” refers to the ability of a nucleic acid molecule to hybridize to at least approximately 6, 12, 20, 30, 50, 100, 150, 200, 300, 350, 400 or 425 consecutive nucleotides of a nucleic acid.

The term “statistically significant” as used herein refers to a measurement which is not the result of random variation or sampling error. For example, the expression “statistically significant change in bioactivity” refers to an increase or decrease of at least about 50% in the value of a particular bioactivity measurement. The bioactivity measurement may refer to, for example, a rate of catalysis or a phenotypic measure of biological complementation. For example, statistically significant increases in growth on galactose (as reflected e.g. by colony size) of a yeast gal4 GAL4:intein strain in contact with a test compound (as compared to growth in the absence of said compound) identify suitable intein self-excision agonists, while statistically significant decreases in growth on galactose of this strain when in contact with a test compound identify suitable intein self-excision antagonists.

The term “target cell” refers to a cell comprising a target polypeptide, the regulation of the bioactivity of which is desired.

The term “target polypeptide” refers to a polypeptide, the bioactivity of which polypeptide is to be regulated. The target protein may comprise one or more intein sequences.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a nucleic acid encoding a chimeric polypeptide of the invention is under the control of a promoter sequence (or other transcriptional regulatory sequence) which controls the expression of the recombinant gene in a cell-type in which expression is intended.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a target polypeptide or, in the case of anti-sense expression from the transferred gene, the expression of a naturally-occurring form of the target polypeptide is disrupted.

As used herein, the term “transgene” means a nucleic acid sequence (encoding, e.g., a chimeric polypeptide of the invention) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a chimeric polypeptide or other polypeptide of interest. However, transgenic animals in which the recombinant chimeric gene is silent are also contemplated, as for example, the FLP or CRE recombinase dependent constructs. Moreover, “transgenic animal” also includes those recombinant animals in which gene disruption of one or more genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “viral vector” refers to a nucleic acid containing at least a portion of a viral genome sufficient for replication and packaging in the presence of an appropriate helper virus and appropriate cell line or packaging extract. For example, by an “AAV vector” is meant a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

By “virion” or “viral particle” is meant a complete virus particle, such as a wild-type (wt) virus particle (comprising a nucleic acid genome associated with a capsid protein coat), or a recombinant virus particle as described below. For example, by “adenoviral virion” is meant a complete virus particle, such as a wild-type (wt) Ad virus particle comprising an Ad nucleic acid genome associated with an Ad capsid protein coat, or a recombinant AAV virus particle as described below. In this regard, single-stranded AAV nucleic acid molecules of either complementary sense, e.g., “sense” or “antisense” strands, can be packaged into any one AAV virion and both strands are equally infectious.

### 4.3. Polypetides and Nucleic Acids of the Present Invention

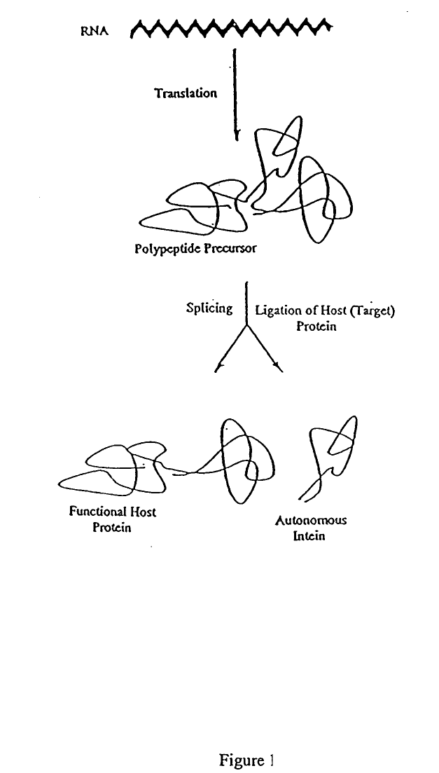

Inteins are a group of related protein elements found within a range of host proteins immediately after their translation. After translation, the intein self-splices itself out of or “autoexcises” itself from the host (target) protein. After autoexcision, the amino-terminal target protein fragment and carboxy-terminal target protein fragment are joined so as to result in a functional target protein and an autonomous intein (see FIG. 1). These amino- and carboxy-terminal fragments of the host protein that become part of the mature functional protein are frequently referred to as “exteins”, and the extein fragment that is C-terminal to the end of the intein is referred to as the C-extein and the amino-terminal fragment that is to the N-terminal side of the intein is referred to as N-extein. There are at least forty known naturally occurring inteins. In fact, these inteins have been compiled in a comprehensive on-line database by the New England Biolabs (http://www.neb.com/neb/inteins.html).

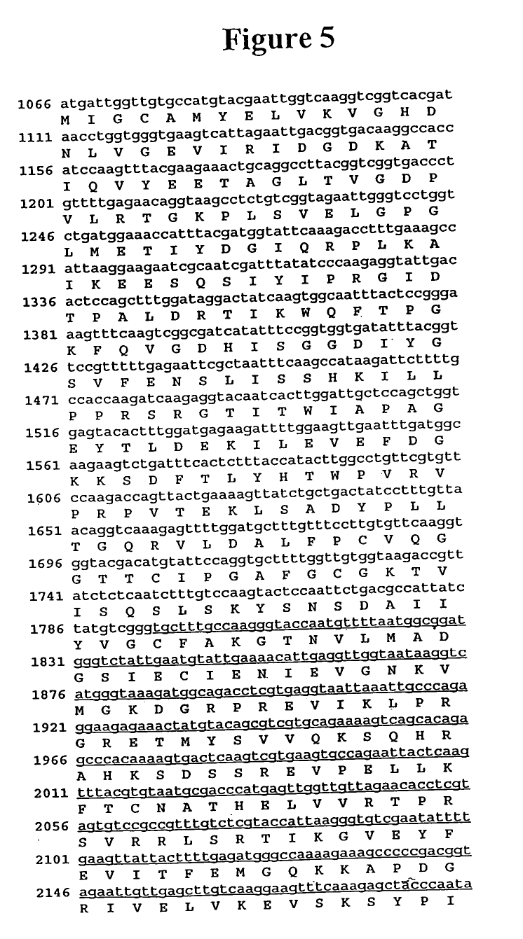

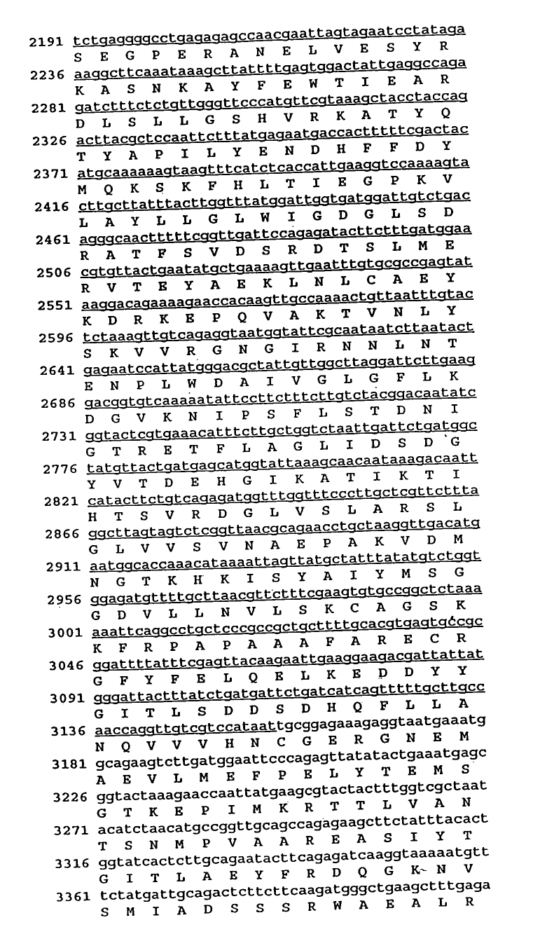

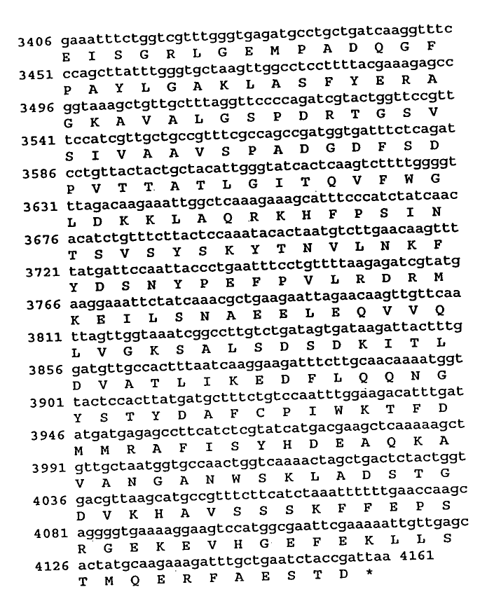

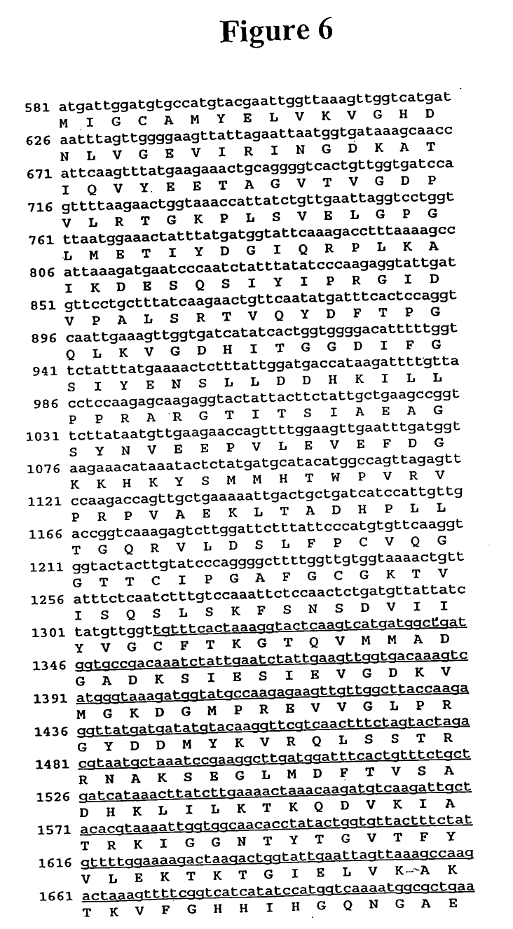

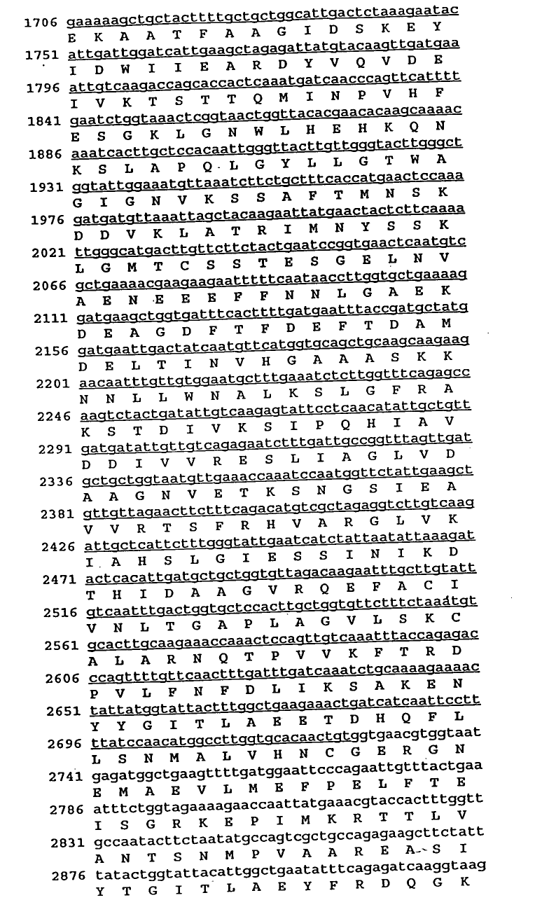

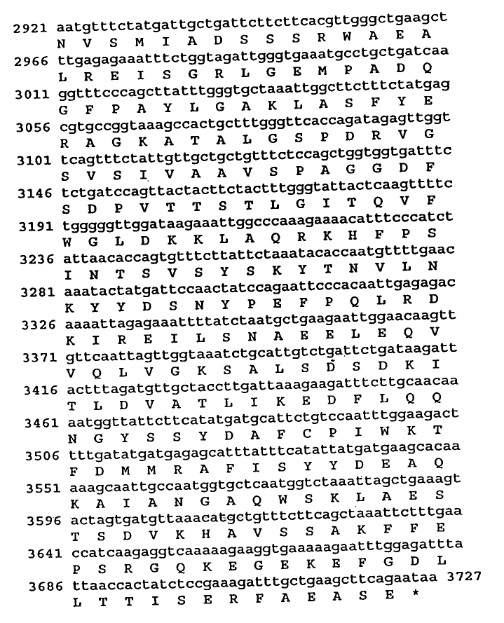

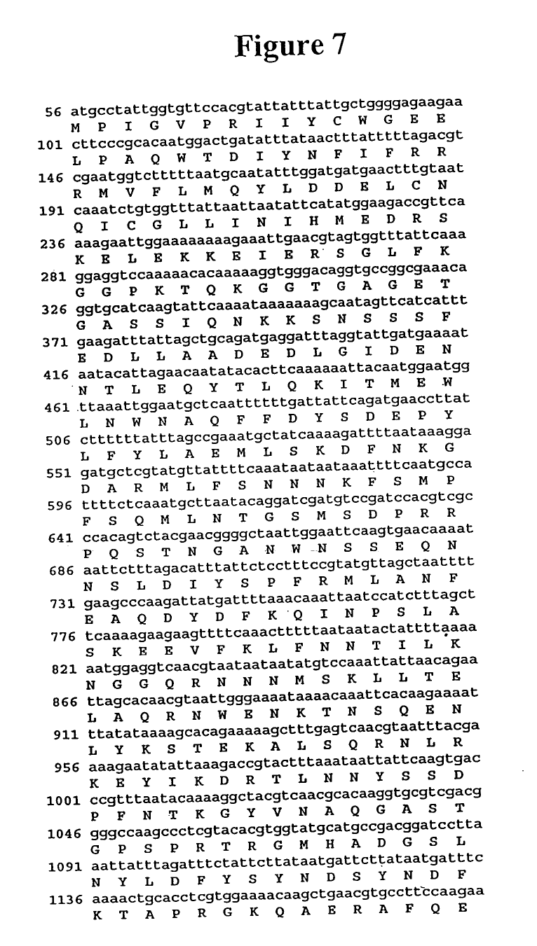

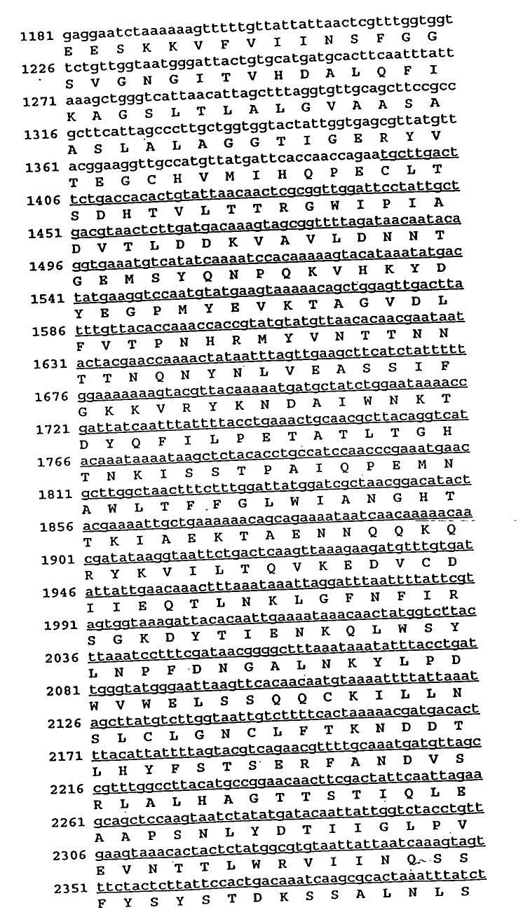

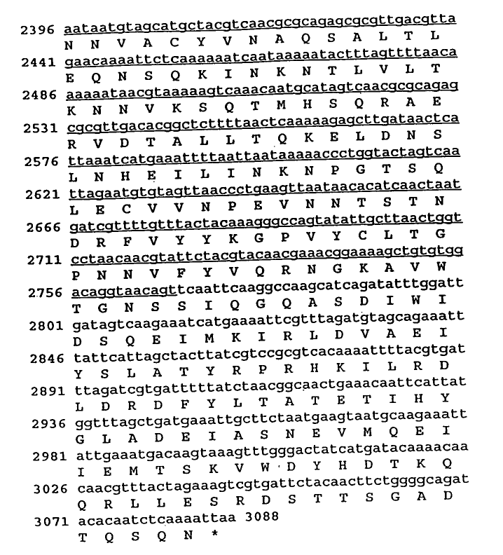

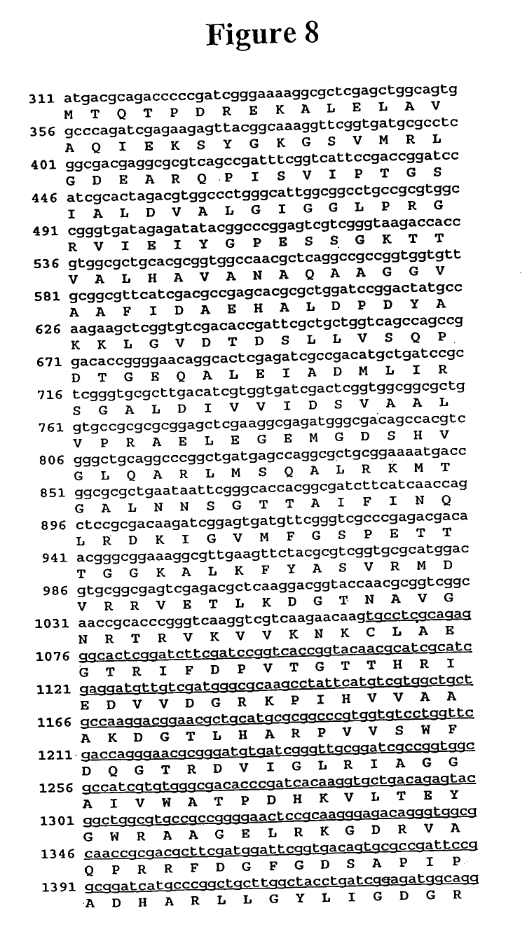

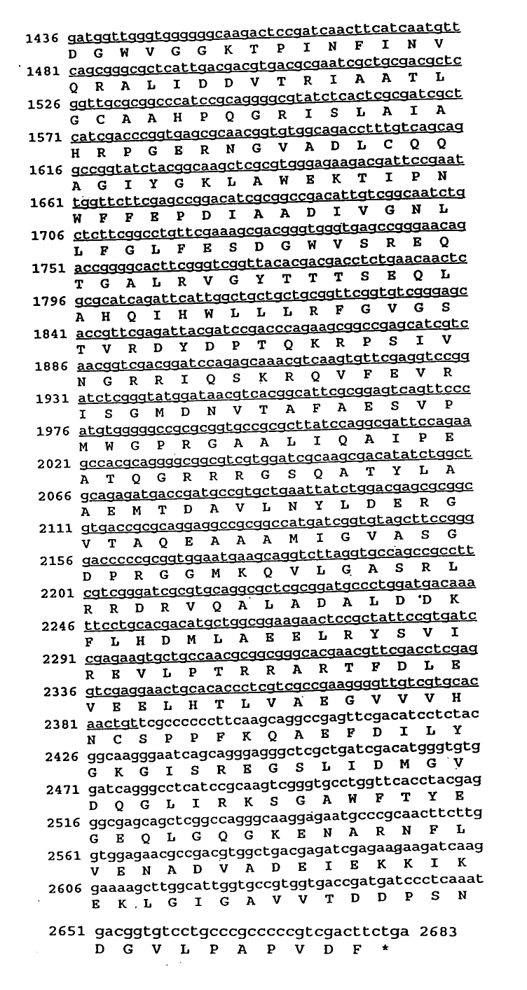

The inteins of this invention may be at least about 100-500 amino acids in length. In one embodiment, the intein is about 450 amino acids in length. In another embodiment, the intein is about 400 amino acids in length. In yet another embodiment, the intein is about 300 amino acids in length. In yet another embodiment, the intein is about 250 amino acids in length. In another embodiment, the intein is about 200 amino acids in length, or about 150 amino acid residues in length, or 100 amino acid residues in length. In a preferred embodiment, the intein is about 105 amino acids in length. Exemplary inteins of this invention include but are not limited to: the Sce VMA intein as shown in FIG. 5 (S. Cerevisiae, Vacuaolar ATPase subunit; GenBank Accession No. M21609) and corresponding to the polypeptide of SEQ ID No. 14 which is encoded by the nucleic acid of SEQ ID No. 13.; Ctr VMA intein as shown in FIG. 6 (Candida Tropicalis Vacuaolar ATPase subunit; GenBank Accession No. M64984) and corresponding to the polypeptide of SEQ ID No. 16 which is encoded by the nucleic acid of SEQ ID No. 15; Ceu clpP intein as shown in FIG. 7 (Chlamydomonas eugametos; GenBank Accession No. L29402) and corresponding to the polypeptide of SEQ ID No. 18 which is encoded by the nucleic acid of SEQ ID No. 17; and the Mtu recA intein as shown in FIG. 8 (Mycobacterium tuberculosis recA intein-containing gene, GenBank Accession No. X58485) and corresponding to the polypeptide sequence of SEQ ID No. 20 which is encoded by the nucleic acid sequence of SEQ ID No. 19.

In one embodiment, the inteins of this invention include a polypeptide which by a nucleotide sequence that hybridizes under stringent conditions to a nucleic acid sequence represented in one or more of SEQ ID Nos. 13, 15, 17 or 19. Appropriate stringency conditions which promote DNA hybridization, for example, 6.0× sodium chloride/sodium citrate (SSC) at about 45 [symbol] C, followed by a wash of 2.0×SSC at 50 [symbol] C, are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50 [symbol] C to a high stringency of about 0.2×SSC at 50 [symbol] C. In addition, the temperature in the wash step can be increased from low stringency conditions at room temperature, about 22 [symbol] C, to high stringency conditions at about 65 [symbol] C.

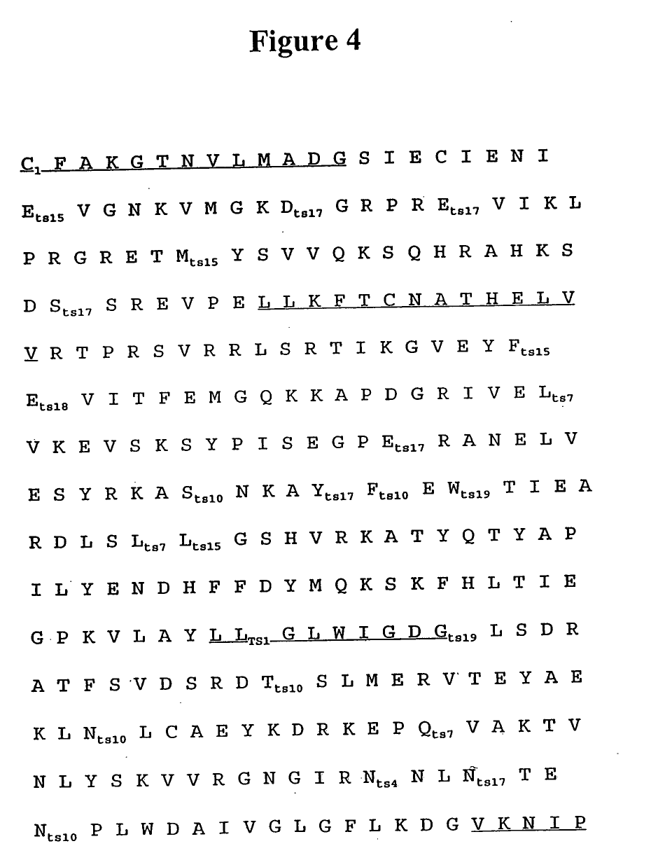

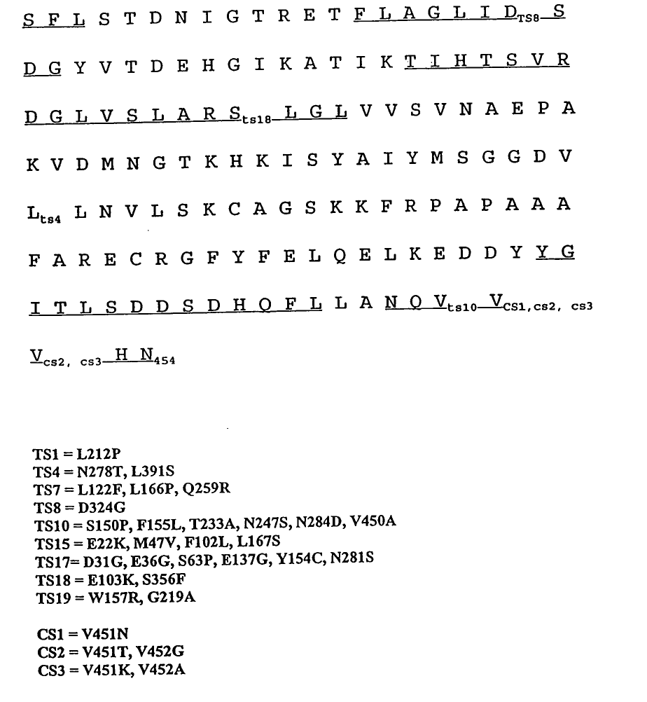

In preferred embodiments the intein of the present invention is a conditional intein allele corresponding to an alteration of the “wild-type” Sce VMA intein shown in FIG. 4 (SEQ ID No. 1). For example, preferred inteins of the invention comprise at least one of the amino acid alterations associated with the temperature sensitive (TS) inteins TS1, TS4, TS7, TS8, TS10, TS15, TS17, TS18 or TS19 or the cold sensitive (CS) intein CS1, CS2 or CS3 as shown in FIG. 4. In certain embodiments, the subject inteins correspond to the conditional alleles of the Saccharomyces cerevisiae VMA intein polypeptide sequence specified by SEQ ID Nos. 2-12. These amino acid alterations can be effected by site-directed mutagenesis of the Sce VMA intein-encoding nucleic acid sequence shown in FIG. 5 (SEQ ID No. 13) in view of the standard genetic code shown below.

AAs = FFLLSSSSYY**CC*WLLLLPPPPHHQQRRRRIIIMTTTTNKKSSRRVVVVAAAADDEEGGGG
Starts = ---M--------------M---------------------------M
Base1 = TTTTTTTTTTTTTTTTCCCCCCCCCCCCCCCCAAAAAAAAAAAAAAAAGGGGGGGGGGGGGGGG
Base2 = TTTTCCCCAAAAGGGGTTTTCCCCAAAAGGGGTTTTCCCCAAAAGGGGTTTTCCCCAAAAGGGG
Base3 = TCAGTCAGTCAGTCAGTCAGTCAGTCAGTCAGTCAGTCAGTGAGTCAGTGAGTCAGTCAGTCAG

For example, the conditional intein TS1, corresponding to a leucine to proline alteration at Sce VMA amino acid residue 212, can be produced by mutating the codon CTT, which occurs beginning at nucleotide 1363 of SEQ ID No. 13, to CCT by a single C to T transition mutation effected through site-directed mutagenesis techniques which are known in the art (see e.g. Costa et al. (1996) Methods Mol. Biol. 57: 239-48).

In certain embodiments, the invention provides controllable intein-encoding nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferbly 95% and even more preferably at least 99% homologous with a nucleotide sequence of an intein-encoding element, e.g., such as a sequence shown in one of SEQ ID Nos: 13, 15, 17 or 19 or complement thereof. In preferred embodiments, of the intein-encoding nucleic acids having ATCC Designation No. ______, corresponding to TS1, ATCC Designation No. ______, corresponding to TS4, ATCC Designation No. ______, corresponding to TS8, ATCC Designation No. ______, corresponding to TS10, ATCC Designation No. ______, corresponding to TS15, ATCC Designation No. ______, corresponding to TS17, ATCC Designation No. ______, corresponding to TS18, ATCC Designation No. ______, corresponding to TS19, ATCC Designation No. ______, corresponding to CS1, ATCC Designation No. ______, corresponding to CS2 or ATCC Designation No. ______, corresponding to CS3. In preferred embodiments, the nucleic acid is from Saccharomyces cerevisiae and in particularly preferred embodiments, the nucleic acid comprises an insertion of the Sca VMA intein into the GAL4 coding sequence immediately before the third cysteine residue within the GAL4 DNA binding domain (GAL4 amino acid residue 20) and having the ATCC deposit Designation No. ______.

In certain embodiments, the allelic changes associated with multiple temperature sensitive alterations can be recombined into a single conditional intein polypeptide. For example the TS 1 allele corresponding to L212P described above can be combined with the amino acid alteration associated with the TS8 allele to yield an L21P, D324G double mutant conditional intein.

The present invention also provides probes/primers comprising a substantially purified oligonucleotide, wherein the oligonucleotide comprises a region of nucleotide sequence which hybridizes under stringent conditions to at least 10 consecutive nucleotides of sense or antisense sequence of one of SEQ ID Nos. 1 or naturally occurring mutants thereof. In preferred embodiments, the probe/primer further comprises a label group attached thereto and able to be detected, e.g. the label group is selected from a group consisting of radioisotopes, fluorescent compounds, enzymes, and enzyme co-factors.

In a further embodiment, the nucleic acid probe hybridizes under stringent conditions to a nucleic acid corresponding to at least 12 consecutive nucleotides of at least one of SEQ ID Nos. 13, 15, 17, 19 or 21; more preferably to at least 20 consecutive nucleotides of SEQ ID Nos. 13, 15, 17, 19 or 21; more preferably to at least 40 consecutive nucleotides of SEQ ID Nos. 13, 15, 17, 19 or 21.

In general, inteins contain about 10 conserved motifs, and these intein motifs can be grouped in three domains according to their location and inferred function. See Peitrokovski, (1998) Protein Science, 7:64-71). These include a N-terminal domain, a C-terminal domain, and an endonuclease EN domain. The N- and C-domains are required for the self-splicing activity and the endonuclease domain is not required for this activity.

The N-domain includes six motifs and spans about 90-150 amino acids. Within the N-domain, domains N2 and N4 are similar to each other and their main attribute is a conserved acidic residue usually preceded by a glycine. Motif N4 is more conserved that motif N2, being longer and less diverse. Nevertheless, the N2 motif is reliably assigned (P value 1·10⁻¹⁷; Schuler et al., 1991) and can be identified in almost all inteins. Motif N4 could not be identified in three of the four eukaryotic inteins, in inteins Tli pol-2, Mja pol-1, and their alleles, and in intein Mja PEPSyn.

The C-domain includes two motifs in the C-terminal spanning about 25-60 amino acids. A central EN-domain typically consisting of four motifs. This domain is about 190-420 amino acids in size and is optional as far as splicing is concerned. Until now, this domain was only known to include motifs similar to those of dodecapeptide (DOD, LAGLI-DADG) homing endonucleases (Pietrokovski (1994) Protein Sci 3: 2340-50; Pietrokovski (1998) Protein Sci 7: 64-71; Perler et al. (1997) Nucleic Acids Res. 25: 1087-93). The central endonuclease domain is separated from the minimal splicing domains by variable spacers, for example, various peptide linkers.

Examples of conserved intein motifs are shown in the table below, this example includes the conserved motifs present in Sce. VMA:

TABLE 1
Conserved Motifs Found In Inteins

| Domain | Conserved Motif | |
|---|---|---|
| N1 Domain | CFAKGTNVLMADG; | (SEQ ID NO:23) |
| N2 Domain | IEVGNKV; | (SEQ ID NO:24) |
| N3 Domain | LLKFTCNATHELVV; | (SEQ ID NO:25) |
| N4 Domain | WKLIDEIKPGDYAVLQ; | (SEQ ID NO:26) |
| EN1 Domain | LLGLWIGDG; | (SEQ ID NO:27) |
| EN2 Domain | VKNIPSFL; | (SEQ ID NO:28) |
| EN3 Domain | FLAGLIDSDG; | (SEQ ID NO:29) |
| EN4 Domain | TIHTSVRDGLVSLARSLGL | (SEQ ID NO:30) |
| C1 Domain | NQVVVHNC. | (SEQ ID NO:31) |
| C2 Domain | YGITLSDDSDHQFL | (SEQ ID NO:32) |

In addition, variant forms, e.g. mutants of the subject inteins are also contemplated as being equivalent to those peptides and DNA molecules that are set forth in more detail, as will be appreciated by those skilled in the art. For example, it is reasonable to expect that an isolated replacement of a leucine with an isoleucine or valine, an aspartate with a glutamate, a threonine with a serine, or a similar replacement of an amino acid with a structurally related amino acid (i.e. conservative mutations) will not have a major effect on the self-splicing activity of the resulting intein polypeptide. In any event, the residues which are essential for splicing are set forth in the section below.

Conservative replacements are those that take place within a family of amino acids that are related in their side chains. Genetically encoded amino acids are can be divided into the following families: (1) acidic (a)=aspartate, glutamate; (2) basic (b)=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine; alternatively serine, threonine and cysteine may be classified separately as being polar amino acids (p); (5) Phenylalanine, tryptophan, and tyrosine are sometimes classified jointly as aromatic amino acids (r); and (6) hydrophobic (h)=glycine, alanine, valine, leucine, isoleucine, and methionine.

In similar fashion, the amino acid repertoire can be grouped as: (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur -containing=cysteine and methionine. (see, for example, Biochemistry, 2nd ed, Ed. by L. Stryer, WH Freeman and Co.: 1981). Whether a change in the amino acid sequence of a peptide results in a functional homolog can be readily determined by assessing the ability of the variant peptide to produce a response in cells in a fashion similar to the wild-type protein.

Furthermore, based upon sequence alignment of various intein polypeptides known in the art, the conserved blocks, may be represented by the following general formulas:

TABLE 2
General Formula for the Conserved Motifs Found In Inteins

| Domain | Conserved Mohf | |
|---|---|---|
| N1 Domain | CX₁X₂X₃DX₄X₅X₆X₇X₈X₉X₁₀G; | (SEQ ID NO:33) |
| N2 Domain | X₁₁X₁₂X₁₃GX₁₄X₁₅V; | (SEQ ID NO:34) |
| N3 Domain | GX₁₆X₁₇X₁₈X₁₉X₂₀TX₂₁X₂₂HX₂₃X₂₄X₂₅X₂₆; | (SEQ ID NO:35) |
| N4 Domain | WX₂₇X₂₈X₂₉X₃₀X₃₁X₃₂X₃₃X₃₄X₃₅DX₃₆X₃₇X₃₈X₃₉X₄₀; | (SEQ ID NO:36) |
| EN1 Domain | LX₄₁GX₄₂X₄₃X₄₄X₄₅X₄₆G; | (SEQ ID NO:37) |
| EN2 Domain | X47KX48IPX49X50X51; | (SEQ ID NO:38) |
| EN3 Domain | X52LX53GX54FX55X56DG; | (SEQ ID NO:39) |
| EN4 Domain | X57X585X59X60X61X62X63X64X64X66X67LLX68X69X70GI | (SEQ ID NO:40) |
| C1 Domain | X71VYDLX72VX73X74X75X76X77FX78. | (SEQ ID NO:41) |
| C2 Domain | NGX79X80X81HNX82 | (SEQ ID NO:42) |

“X” is an amino acid which can be selected from amongst amino acid residue which would be conservative substitutions for the amino acids which appear naturally in each of those positions. For instance, conserved block N1 comprises the following amino avid residues: X1 belongs to class h as designated above, X2 and X3 can be any amino acid, X4 belongs to class p, X5 may be any amino acid, X6, X7, and X8 belong to class h, X9, X10 may be any amino acid.

Conserved block N2 comprises X11 which belong to class h, X12 belongs to class b, X13 belongs to class h, X14 belongs to class a, and X15 may be any amino acid.

Conserved block N3 comprises X16 and X17 which may be any amino acid, X18 belongs to class h, X19 may be any amino acid, X20 belongs to class h, X21, X22, and X23 may be any amino acid, X24, X25, and X26 are class h.

Conserved block N4 comprises X27 through X29, X3 1, X33 through X40 may be any amino acid, X30 belongs to class a, and X32 is class h.

Conserved block EN1 comprises X41 which belongs to class h, X42 and X43 may be any amino acid, X44 and X45 are h, X46 is class a.

Conserved block EN2 comprises X47 through X50 which may be any amino acid, X51 is class h.

Conserved block EN3 comprises X52 and X53 which may be any amino acid, X54 is class h, X55 is class a, and X56 is class h.

Conserved block EN4 comprises X57 which belongs to class b, X58 through X60 may be any amino acid, X 61 and X62 are class h, X63 and X64 may be any amino acid, X65 is class h, X66 through X69 may be any amino acid and X70 is class h.

Conserved block C1 comprises X71 which belongs to class r, X72 is a member of class p, X73 is class a, X74 through X77 may be any amino acid, X78 is class h.

Conserved block C2 comprises X79, X80, and X81 are class h, and X82 is class p.

In one embodiment, the invention includes a nucleic acid probe which hybridizes under stringent conditions to a nucleic acid corresponding to SEQ ID Nos. 13, 15, 17, 19 or 21; more preferably to at least 20 consecutive nucleotides of SEQ ID Nos. 13, 15, 17, 19 or 21; more preferably to at least 40 consecutive nucleotides of SEQ ID Nos. 13, 15, 17, 19 or 21.

In one embodiment, this invention includes within its scope condition-sensitive mutant inteins. A conditional mutant intein retains its function, i.e., the self-splicing function, under one set of conditions, called permissive, but lacks that function under a different set of conditions, called nonpermissive; the latter must still be permissive for the wild-type allele of the gene. Conditional mutants are presumed, in most cases, to result from missense mutations in a structural gene encoding a protein. In the case of temperature-sensitive (ts) mutants, the amino acid replacement resulting from the missense mutation partially destabilizes the encoded protein, resulting in the maintenance of its three-dimensional integrity only at relatively low temperatures.

Several types of conditional mutants and methods for producing them have been developed since the original demonstration of the utility of ts mutants (Horowitz, Genetics 33, 612 (1948). Accordingly, this invention provides a means for generating conditional mutants of any gene product of interest without having to laboriously screen for mutations within the host itself.

In certain embodiments, the condition-sensitive mutant intein is temperature sensitive (TS) or cold sensitive (CS) intein. In alternative embodiments, the condition-sensitive mutant intein is sensitive to one or more of pH, exposure to light, unblocking of amino acid residues by dephosphorylation or deglycosylation, ionic concentrations, concentration of various metals, osmolarity, and/or the presence or absence of certain exogenous chemical agents. Examples of exogenous chemicals include agents such as rapamycin or rapamycin analogs useful in mammalian systems and chemicals such as salicylic acid, abscissic acid useful in plant systems. Other examples of an exogenous chemical signalling agent of the present invention include oligonucleotides such as double-stranded nonhydrolyzable synthetic oligonculeotides which are recognized by an endonuclease catalytic site encoded by the regulatable intein of the invention.

In one embodiment, the temperature sensitive mutant inteins are those which do not undergo self-excision from the target protein at temperatures over about 29° C. In another embodiment, the cold-sensitive mutant inteins are those that do not undergo self-excision at temperatures below 18° C. Preferably, predetermined excision conditions are experimentally determined taking into consideration temperatures at which the target protein will denature or undergo thermal inactivation. Examples of these conditional mutants include temperature sensitive and cold sensitive alleles of the Sce. VMA intein. The specific amino acid changes in these alleles due to these specific mutations are listed in the table below:

TABLE 3
Condition-Sensitive Mutations

| Sce. VMA Allele | Amino Acid Change |
|---|---|
| TS1 | L212P |
| TS4 | N278T, L391S |
| TS7 | L122F, L166P, Q259R |
| TS8 | D324G |
| TS10 | S150P, F155L, T233A, N247S, |
| | N284D, V450A |
| TS15 | E2K, M47V, F102L, L167S |
| TS17 | D31G, E36G, S63P, E137G, Y154C, |
| | N281S |
| TS18 | E103K, S356F |
| TS19 | W157R, L219A |
| CS1 | V451N |
| CS2 | V451T, V452G |
| CS3 | V451K, V452A |

In one embodiments the condition-sensitive mutant inteins of this invention include a polypeptide which is encoded by a nucleotide sequence that hybridizes under stringent conditions to a nucleic acid sequence represented in one or more of SEQ ID Nos. 13, 15, 17, 19or21.

The present invention also provides probes/primers comprising a substantially purified oligonucleotide, wherein the oligonucleotide comprises a region of nucleotide sequence which hybridizes under stringent conditions to consecutive nucleotides of sense or antisense sequence of SEQ ID Nos. 13, 15, 17, 19 or 21, or naturally occurring mutants thereof. In preferred embodiments, the probe/primer further comprises a label group attached thereto and able to be detected, e.g. the label group is selected from a group consisting of radioisotopes, fluorescent compounds, enzymes, and enzyme co-factors.

In another embodiment, the inteins of this invention include polypeptide sequences comprising only the N-and C-domains, which are required for the efficient self-splicing of the intein. Thus, this invention includes inteins comprising the minimal portions required for self-splicing, for example these include inteins comprising mainly the N and C domains together with a minimal linker, such that, the linker provides the flexibility required for proper protein-folding and consequently proper intein self-splicing.

The N domain may be about 90-150 amino acids in length. In one embodiment, the N domain is about 130 amino acids in length. In another embodiment, the N domain is about 100 amino acids in length. In yet another embodiment, the N domain is about 95 amino acids in length. In a preferred embodiment, the N domain is about 90 amino acids in length.

The C domain may be at least 35-55 amino acids in length. In one embodiment the C domain is about 50 amino acids in length. In another embodiment, the C domain is about 40 amino acids in length, and in a preferred embodiment, the C domain is about 35 amino acids in length.

These minimal inteins may be generated by deleting the central region encoding the entire endonuclease region. For example, Shingledecker et al. (Gene 207:187-195 (1998), have shown that a functional intein was formed by the deletion of the entire endonuclease domain from the Mycobacterium tuberculosis recA intein, wherein the deletion resulted in an intein comprising the N and C domains together with a undecapeptide spacer.

In another embodiment, this invention includes inteins wherein either the N and/or the C domains are synthesized separately and reconstituted to provide a self-splicing intein. The N and C domains may either be isolated and purified or may be synthesized. In addition, these domains may be from the same or different target (host) polypeptides. In one embodiment, the invention also includes within its scope a N-extein-N-intein fragment which may be expressed in cells and a C-intein-C-extein fragment, which may be independently expressed in cells, wherein interaction of the two fragments yields an full length N-extein-N-intein-C-intein-C-extein polypeptide product.

In another aspect, the invention also includes a N-extein-N-intein-L (ligand) fragment which may be expressed in cells and a LBD (ligand binding domain)-C-intein-C-extein fragment, which may be independently expressed in cells, wherein interaction between the ligand and the ligand binding domains of the two fragments yields an full length N-extein-N-intein-L-LBD-C-intein-C-extein polypeptide product. Examples of suitable ligands and ligand binding domains, include but are not limited to polypeptides such as FK506 binding proteins/RAP-binding proteins, and antibody/hapten pairs. A skilled artisan can readily adapt any known protein binding domain/ligand pair for use in the present methods. Further, as will be evident to the skilled artisan, the ligand and the ligand binding domain may be interchangeably present on either fragment described herein.

Formation of the full length N-extein-N-intein-C-intein-C-extein polypeptide or the N-extein-N-intein-L-LBD-C-intein-C-extein polypeptide product is followed by excision of the intein to produce a functional target protein.

In one aspect of this invention, either the formation of the full length polypeptide or the splicing of the intein after the formation of the full length polypeptide may be subject to exogenous regulation.

The linker used herein may be any linker which provides the flexibility required for the formation of the splicing active site required for proper folding of the intein to bring together the two splice junctions, and other amino acid residues which may assist in the splicing reaction. This linker can facilitate enhanced flexibility of the intein allowing the N- and C- domains to freely and (optionally) simultaneously interact by reducing steric hindrance between the two fragments, as well as allowing appropriate folding of each portion to occur. The linker can be of natural origin, such as a sequence determined to exist in random coil between two domains of a protein. Alternatively, the linker can be of synthetic origin.

In one embodiment, the linker may be a peptide linker, for instance, the linker may be a poly-glycine linker, or a linker containing Asn-Gly repeats, or Gly-Ser repeats. In a preferred embodiment the linker is a (Gly4Ser)3 sequence. Peptide linkers may be between about 5-50 amino acids, more preferably the linker is 5-30 amino acids in length and most preferably the linker is 6-20 amino acid residues in length. Linkers of this type are described in Huston et al. (1988) PNAS 85:4879; and U.S. Pat. Nos. 5,091,513 and 5,258,498. Naturally occurring unstructured linkers of human origin are preferred as they reduce the risk of immunogenicity.

This invention further contemplates a method for generating sets of combinatorial mutants of the subject intein proteins as well as truncation mutants, and is especially useful for identifying potential variant sequences (e.g., homologs). The purpose of screening such combinatorial libraries is to generate, for example, novel conditional intein equivalents which can be used in the method of the present invention. For example, the combinatorially-derived homologs can be generated to have an increased sensitivity of regulation relative to a given intein conditional allele. Alternatively, the combinatorially-derived conditional intein homolog may correspond to an altered nucleic acid sequence which, for example, facilitates cloning into a target gene or which alters codon utilization to correspond to a more preferred set of codons for a given organism in which the regulated target gene is to be expressed (for review of organismal codon bias see e.g. Sharp et al. (1988) Nucleic Acids Res. 16: 8207-11).

In one embodiment, the variegated library of intein variants is generated by combinatorial mutagenesis at the nucleic acid level, and is encoded by a variegated gene library. For instance, a mixture of synthetic oligonucleotides can be enzymatically ligated into gene sequences such that the degenerate set of potential Intein sequences are expressible as individual polypeptides, or alternatively, as a set of larger fusion proteins (e.g., for phage display) containing the set of intein sequences therein.

There are many ways by which such libraries of potential intein homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential Intein sequences. The synthesis of degenerate oligonucleotides is well known in the art (see for example, Narang, S A (1983) Tetrahedron 39:3; Itakura et al. (1981) Recombinant DNA, Proc 3^{rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for an intein clone in order to generate a variegated population of intein fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of an intein coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of intein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate intein sequences created by combinatorial mutagenesis techniques. Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

### 4.4. Modification of Target Genes and Polypeptides

The invention provides methods by which a target polypeptide which encodes at least one bioactivity can be modified by the insertion of a regulatable intein such that the bioactivity becomes controllable by regulating the excision of the regulatable intein. We provide herein specific examples in which a target polypeptide, selected by virtue of its encoded bioactivity, is modified by the insertion of such a regulatable intein sequence (see Examples). General considerations to be made by the skilled artisan when engineering the target polypeptide::intein hybrid are discussed below. Further minor considerations will be obvious to those of skill in the art.

The sequence of naturally occurring intein containing gene sequences, along with various mechanistic studies on intein excision, provides guidance for the modification of a target polypeptide with a regulatable intein. For example, the inserted intein open reading frame (ORF) must be “in frame” with the target polypeptide at the point of insertion in order that a full-length target polypeptide::intein of the general structure N-Extein target polypeptide-intein-C-extein target polypeptide can be made. The reading frame must be retained across both the N-extein/intein junction and the intein/C-extein junction.

Alternatively, two separate hybrid polypeptides corresponding to a first N-Extein target polypeptide-N-terminal-intein polypeptide and a second C-terminal-intein-C-terminal-extein polypeptide can be engineered so that regulatable trans-splicing auto-excision event results in the joining of the N-Extein and C-Extein polypeptide segments to produce a trans-spliced target polypeptide. In this embodiment, the N-extein/intein junction and the intein/C-extein junction are each engineered separately, but nevertheless must each be made to retain the existing reading frame across each polypeptide junction.

A second consideration for the site of insertion into the target polypeptide of the regulatable intein sequence is selection of a site adjacent to a target polypeptide hydroxyl or thiol moiety such as provided by the amino acid side chain of a serine, threonine or cysteine residue. Polypeptide sequence alignments of naturally-occurring intein-containing gene products reveals the existence of a conserved serine, threonine or cysteine at the site of insertion into the host protein (Perler F B, et al. (1997) Nucleic Acids Res. 25:1087-93). Furthermore, mutagenesis of this conserved serine, threonine or cysteine at the intein-C-extein junction resulted in loss of intein autoexcision activity (Hirata et al. (1992) Biochem. Biophys. Res. Commun. 188: 40-47; Cooper et al. (1993) EMBO J 12: 2575-83; Davis et al. (1992) Cell 71, 201-10). Certain studies have suggested that the identity of the amino-terminal residue of the intein, which is also a conserved serine, threonine or cysteine, should match that of this conserved amino terminal residue of the C-extein- particularly when the amino-terminal intein residue is a cysteine (Chong et al. (1996) J Biol. Chem. 271: 22159-68). Therefore, in preferred embodiments, the conditional intein polypeptide is inserted upstream (amino-terminal) to a cysteine, serine or threonine, the identity of which matches that of the amino-terminal residue of the selected intein. This limitation on the site of intein insertion into the host polypeptide should not prove limiting however, as serine, threonine and cysteine collectively account for well over ten percent of the total amino acid composition of a number of representative proteins (Lehninger (1976) Worth Publishers, Inc., p. 101). Therefore, by selection of an appropriate conditional intein, virtually any target polypeptide can be modified an endogenous serine, threonine or cysteine residue to yield a target polypeptide::intein hybrid gene product from which, under appropriate conditions, the endogenous auto-excision activity of the intein can be activated and the inserted intein sequence thereby excised from the target polypeptide. Furthermore, in order for the inserted conditional intein to exert control of a bioactivity of the target polypeptide, in preferred embodiments, the site of insertion of the intein polypeptide must be selected so as to interfere with the bioactivity when the intein is present in the target::intein hybrid. Guidance in constructing such a hybrid are provided above.

In certain specialized embodiments of the invention, the target polypeptide encodes a bioactivity which is partially or completely inactive in the absence of an inserted intein. Such target polypeptides may correspond, for example, to the fusion of two polypeptides which interact with one another to produce a measurable bioactivity but which are fused in such close proximity (e.g. directly abutting the polypeptide domains or fusing them with only a short linker polypeptide) as to cause a steric inhibition of their interaction. In this particular instance, the insertion of an heterologous regulatable intein sequence between the two domains causes an increase in the bioactivity resulting from the appropriate and sterically proper interaction of the two target polypeptides. This particular embodiment of the invention allows for the regulation of the target polypeptide in a manner opposite that of the preferred embodiment discussed above—that is, signals which increase the self-excision of the inserted intein (such as intein self-excision agonist compounds) actually decrease the target polypeptide bioactivity whereas signals which decrease the self-excision of the inserted intein (such as intein self-excision antagonist compounds) actually increase the target polypeptide bioactivity.

### 4.5. Methods of Preparing Target:Intein Hybrid Polypeptides

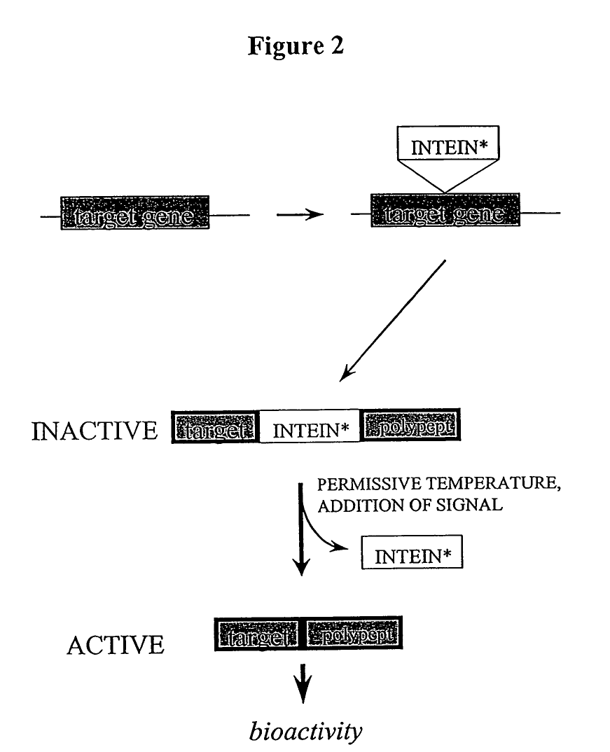

The Intein-target hybrids may be prepared by the methods which are well known in the art. The method contemplates both in vivo and in vitro methods for creating these hybrids. In preferred embodiments a nucleic acid encoding a regulatable intein is inserted into a nucleic acid which encodes a target polypeptide as shown in FIG. 2. General cloning techniques (see e.g. Sambrook et al. (1989) Molecular Cloning: A Laboratory Manual (Cold Spring Harbor Press)) can be used in the method of the invention to obtain suitable target gene:intein hybrid nucleic acids of the invention. The invention provides other techniques particularly well suited to the insertion of the regulatable intein-encoding nucleic acid sequence into the target polypeptide-encoding nucleic acid sequence while retaining the correct reading frame of the target gene at both the upstream and downstream insertion junctions. Attention to the reading frame of the target gene allows recombinant production of the target polypeptide:Intein hybrid polypeptide.

For example, in one aspect, the method includes a PCR-based approach called splicing by overlap extension (SOE) which is not sequence-dependent and does not depend on the occurrence of restriction enzyme recognition sequences at the recombination site. Gene splicing by overlap extension is an effective way for recombining DNA molecules at precise junctions irrespective of nucleotide sequences at the recombination site and without the use of restriction endonucleases or ligase. Fragments from the genes that are to be recombined are generated in separate polymerase chain reactions (PCRs). The primers are designed so that the ends of the products contain complementary sequences. When these PCR products are mixed, denatured, and reannealed, the strands having the matching sequences at their 3′ ends overlap and act as primers for each other. Extension of this overlap by DNA polymerase produces a molecule in which the original sequences are ‘spliced’ together. This technique is used to construct a gene encoding a mosaic protein comprised of an intein and a target polypeptide.

In certain situations, the SOE method of recombining gene sequences is a significant improvement over standard techniques. This method is particularly useful when sequences must be precisely joined within a very limited region. In addition to being an improved method for recombining DNA, SOE allows site-directed mutagenesis to be performed simultaneously with recombination. The product in a SOE reaction is a mosaic of natural sequences connected by synthetic regions, and the sequence of these synthetic regions is entirely at the discretion of the genetic engineer.

### 4.6. Agonist and Antagonist Signals of the Invention

The invention further provides signals which are used to regulate the self-excision activity of an intein polypeptide. In general, the selection of a signal is predicated upon the nature of the intein to be regulated. For example, self-excision of the temperature-sensitive conditional inteins can be antagonized by increasing the temperature, while self-excision of the cold-sensitive conditional inteins can be antagonized by decreasing the temperature. In contrast, the trans-spliced regulatable inteins described herein can be agonized by the addition of an exogenous chemical dimerizer such as rapamycin. Each of these examples entail the use of a genetically modified intein, however the invention provides methods by which an intein which has not been genetically modified can be regulated by means of an appropriate agonist or antagonist signal.

For example, many naturally-occurring inteins frequently encode a homing endonuclease activity which recognizes and cleave at a nucleic acid sequence adjacent to the site of its insertion into the host gene. This cleavage event initiates a series of recombinogenic events which can effect the “mobilization” of the intein-encoding sequence. The nucleic acid sequence recognized by the homing endonuclease can thus be identified from the nucleic acid sequence surrounding this junction (see e.g., Nishioka, et al. (1998) Nucleic Acids Res. 26: 4409-12). Therefore a double-stranded oligonucleotide which comprises the minimal recognition sequence for such an endonuclease will therefore bind to a target:intein hybrid polypeptide which carries this endonuclease function. This provides for a readily-identifiable high affinity ligand for use in directly or indirectly regulating an intein self-excision activity. For example, a nonhydrolyzable synthetic oligonucleotide which binds tightly to the intein endonuclease catalytic site but does not undergo hydrolytic chain breakage can be used to antagonize an intein self-excision reaction. Preferably, such a nonhydrolyzable substrate is designed to mimic a substrate transition state which occurs during catalysis. Such transition state analogs frequently bind with extremely high affinities to the corresponding catalytic site and therby inhibit catalysis of the natural substrates. In some embodiments, the formation of an oligonucleotide/intein-endonuclease complex prevents self-excision of the intein from the target polypeptide. In these instances, the synthetic oligonucleotide alone can serve as a signaling agent in the method of the invention. In preferred embodiments, the synthetic oligonucleotide is further modified to include one or more activities which serve to agonize or antagonize the self-excision of the intein. For example, self-excision can be readily antagonized by addition of chemically active amino acid crosslinking groups which, in preferred embodiments, recognize one or more of the amino acid side groups which function in the intein self-excision reaction.

Still other signals of the invention include those which can be identified by routine screening for chemical ligands or inhibitors of intein self-excision using appropriate high-throughput screening techniques.

### 4.7. Nucleic Acid Compositions

In another aspect of the invention, the proteins described herein are provided in expression vectors. For instance, expression vectors are contemplated which include a nucleotide sequence encoding a polypeptide containing a composite activator of the present invention, which coding sequence is operably linked to at least one transcriptional regulatory sequence. Regulatory sequences for directing expression of the instant fusion proteins are art-recognized and are selected by a number of well understood criteria. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the fusion proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast ÿ-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

As will be apparent, the subject gene constructs can be used to cause expression of the subject fusion proteins in cells propagated in culture, e.g. to produce proteins or polypeptides, including fusion proteins, for purification.

This invention also pertains to a host cell transfected with a recombinant gene in order to express one of the subject polypeptides. The host cell may be any prokaryotic or eukaryotic cell. For example, a fusion proteins of the present invention may be expressed in bacterial cells such as E. coli, insect cells (baculovirus), yeast, or mammalian cells. Other suitable host cells are known to those skilled in the art.

Accordingly, the present invention further pertains to methods of producing the subject fusion proteins—e.g., the target polypeptide:intein chimeric polypeptides described herein. For example, a host cell transfected with an expression vector encoding a protein of interest can be cultured under appropriate conditions to allow expression of the protein to occur. The protein may be secreted, by inclusion of a secretion signal sequence, and isolated from a mixture of cells and medium containing the protein. Alternatively, the protein may be retained cytoplasmically and the cells harvested, lysed and the protein isolated. A cell culture includes host cells, media and other byproducts. Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

Thus, a coding sequence for a fusion protein of the present invention can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures.

Expression vehicles for production of a recombinant protein include plasmids and other vectors. For instance, suitable vectors for the expression of the instant fusion proteins include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant fusion proteins by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, e.g., a composite activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ÿCrip, ÿCre, ÿ2 and ÿAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Ophthalmol Vis Sci 35:2662-2666) In addition to viral transfer methods, such as those illustrated above, non-viral methods can also be employed to cause expression of a protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In a representative embodiment, a gene encoding a composite activator can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication W091/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, any of the subject gene constructs can be used to transfect specific cells in vivo using a soluble polynucleotide carrier comprising an antibody conjugated to a polycation, e.g. poly-lysine (see U.S. Pat. No. 5,166,320). It will also be appreciated that effective delivery of the subject nucleic acid constructs via -mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art.

For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

In some embodiments of the invention, the target gene to be regulated by the regulatable intein is an endogenous gene, which contains an exogenous regulatable intein sequence. The exogenous regulatable intein sequence can be inserted into the endogenous gene's coding sequence. In certain embodiments, the endogenous target gene is a DNA binding protein, capable of binding with high affinity and specificity to a target sequence. In a preferred embodiment, the DNA binding protein is human. However, the DNA binding protein can be from any other species. For example, the DNA binding protein can be from the yeast GAL4 protein.

In other embodiments, the target gene to be regulated by the regulatable intein is an exogenous gene. In some embodiments, the exogenous gene is integrated into the chromosomal DNA of a cell. The exogenous gene can be inserted into the chromosomal DNA, or the exogenous gene can substitute for at least a portion of an endogenous gene. Alternatively, the exogenous gene can be present on an extrachromosomal DNA element, such as a plasmid or a viral vector. The target gene can be present in a single copy or in multiple copies. In view of the experimental results described herein, it is not necessary that the target gene be present in more than one copy. However, if even higher levels of protein encoded by the target gene is desired, multiple copies of the gene can be used.

A wide variety of genes can be employed as the target gene, including genes that encode a therapeutic protein. The target gene can be any sequence of interest which provides a desired phenotype. It can encode a surface membrane protein, a secreted protein, a cytoplasmic protein, or there can be a plurality of target genes encoding different products. The proteins which are expressed, singly or in combination, can involve homing, cytotoxicity, proliferation, immune response, inflammatory response, clotting or dissolving of clots, hormonal regulation, etc. The proteins expressed may be naturally-occurring proteins, mutants of naturally-occurring proteins, unique sequences, or combinations thereof.

Various secreted products include hormones, such as insulin, human growth hormone, glucagon, pituitary releasing factor, ACTH, melanotropin, relaxin, etc.; growth factors, such as EGF, IGF-1, TGF-ÿ, -ÿ, PDGF, G-CSF, M-CSF, GM-CSF, FGF, erythropoietin, thrombopoietin, megakaryocytic stimulating and growth factors, etc.; interleukins, such as IL-1 to -13; TNF-ÿ and -ÿ, etc.; and enzymes and other factors, such as tissue plasminogen activator, members of the complement cascade, performs, superoxide dismutase, coagulation factors, antithrombin-III, Factor VIIIc, Factor VIIIvW, Factor IX, ÿ-antitrypsin, proteinC, proteinS, endorphins, dynorphin, bone morphogenetic protein, CFTR, etc.

The gene can encode a naturally-occurring surface membrane protein or a protein made so by introduction of an appropriate signal peptide and transmembrane sequence. Various such proteins include homing receptors, e.g. L-selectin (Mel-14), blood-related proteins, particularly having a kringle structure, e.g. Factor VIIIc, Factor VIIIvW, hematopoietic cell markers, e.g. CD3, CD4, CD8, Bcell receptor, TCR subunits ÿ, ÿ, ÿ, ÿ, CD10, CD19, CD28, CD33, CD38, CD41, etc., receptors such as the interleukin receptors IL-2R, IL-4R, etc., channel proteins, for influx or efflux of ions, e.g. H+, Ca+2, K+, Na+, Cl−, etc., and the like; CFTR, tyrosine activation motif, zap-70, etc.

Proteins may be modified for transport to a vesicle for exocytosis. By adding the sequence from a protein which is directed to vesicles, where the sequence is modified proximal to one or the other terminus, or situated in an analogous position to the protein source, the modified protein will be directed to the Golgi apparatus for packaging in a vesicle. This process in conjunction with the presence of the chimeric proteins for exocytosis allows for rapid transfer of the proteins to the extracellular medium and a relatively high localized concentration.

Also, intracellular proteins can be of interest, such as proteins in metabolic pathways, regulatory proteins, steroid receptors, transcription factors, etc., depending upon the nature of the host cell. Some of the proteins indicated above can also serve as intracellular proteins.

By way of further illustration, in T-cells, one may wish to introduce genes encoding one or both chains of a T-cell receptor. For B-cells, one could provide the heavy and light chains for an immunoglobulin for secretion. For cutaneous cells, e.g. keratinocytes, particularly stem cells keratinocytes, one could provide for protection against infection, by secreting ÿ-, ÿ- or ÿ-interferon, antichemotactic factors, proteases specific for bacterial cell wall proteins, etc.

In addition to providing for expression of a gene having therapeutic value, there will be many situations where one may wish to direct a cell to a particular site. The site can include anatomical sites, such as lymph nodes, mucosal tissue, skin, synovium, lung or other internal organs or functional sites, such as clots, injured sites, sites of surgical manipulation, inflammation, infection, etc. By providing for expression of surface membrane proteins which will direct the host cell to the particular site by providing for binding at the host target site to a naturally-occurring epitope, localized concentrations of a secreted product can be achieved. Proteins of interest include homing receptors, e.g. L-selectin, GMP140, CLAM-1, etc., or addressing, e.g. ELAM-1, PNAd, LNAd, etc., clot binding proteins, or cell surface proteins that respond to localized gradients of chemotactic factors. There are numerous situations where one would wish to direct cells to a particular site, where release of a therapeutic product could be of great value.

For use in gene therapy, the target gene can encode any gene product that is beneficial to a subject. The gene product can be a secreted protein, a membraneous protein, or a cytoplasmic protein. Preferred secreted proteins include growth factors, differentiation factors, cytokines, interleukins, tPA, and erythropoietin. Preferred membraneous proteins include receptors, e.g, growth factor or cytokine receptors or proteins mediating apoptosis, e.g., Fas receptor. Other candidate therapeutic genes are disclosed in PCT/US93/01617.

In yet another embodiment, a “gene activation” construct which, by homologous recombination with a genomic DNA, alters the transcriptional regulatory sequences of an endogenous gene, can be used to introduce recognition elements for a DNA binding activity of one of the subject engineered proteins. A variety of different formats for the gene activation constructs are available. See, for example, the Transkaryotic Therapies, Inc PCT publications WO93/09222, WO95/31560, WO96/2941 1, WO95/31560 and WO94/12650.

### 4.8. Kits

This invention further provides kits useful for the foregoing applications. One such kit contains one or more nucleic acids encoding a chimeric polypeptide comprising a target polyeptide which encodes a bioactivity and a regulatable intein, which is inserted into the target polypeptide. The kit may further comprise an additional nucleic acids such as specialized vectors which contain a cloning site for insertion of a desired target gene by the practitioner. For example, a preferred kit would contain a cloning site comprising at least one restriction site for insertion of an N-Extein of a target polypeptide, which is supplied by the user of the kit. In preferred embodiments, the cloning site is a polylinker. In preferred embodiments, this N-Extein cloning site is followed by a regulatable Intein sequence. In particularly preferred embodiments, the N-Extein cloning site of the vector is made available to the user in all three possible reading frames by supplying three different versions of the vector corresponding to single nucleotide insertions at the cloning site so that an in-frame fusion of the N-Extein to the regulatable Intein occurs. In preferred embodiments, the regulatable Intein sequence is further followed by a cloning site for a C-Extein element of the target sequence, which target may be supplied by the user. In still more preferred embodiments, versions of the vector corresponding to all three possible reading frames between the regulatable intein and the C-extein are made available to the user. For regulatable applications, i.e., in cases in which the recombinant protein contains a ligand binding domain or inducible domain, the kit may further contain an oligomerizing agent, such as the macrolide dimerizers discussed above. Such kits may for example contain a sample of a dimerizing agent capable of dimerizing the two recombinant proteins and activating transcription of the target.

Constructs may be designed in accordance with the principles, illustrative examples and materials and methods disclosed in the patent documents and scientific literature cited herein, each of which is incorporated herein by reference, with modifications and further exemplification as described herein. Components of the constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. In the case of DNA constructs encoding chimeric proteins, DNA sequences encoding individual domains and sub-domains are joined such that they constitute a single open reading frame encoding a chimeric protein capable of being translated in cells or cell lysates into a single polypeptide harboring all component domains. The DNA construct encoding the chimeric protein may then be placed into a vector that directs the expression of the protein in the appropriate cell type(s). For biochemical analysis of the encoded chimera, it may be desirable to construct plasmids that direct the expression of the protein in bacteria or in reticulocyte-lysate systems. For use in the production of proteins in mammalian cells, the protein-encoding sequence is introduced into an expression vector that directs expression in these cells. Expression vectors suitable for such uses are well known in the art. Various sorts of such vectors are commercially available.

### 4.9. Transgenic Organisms

The invention provides transgenic plants and animals which carry one or more intein modified target genes which can be regulated. These transgenic organisms can be generated with the nucleic acid target gene:intein hybrids of the invention. For example, the invention further provides for transgenic animals, which can be used for a variety of purposes, e.g., to study the function of a target gene. The transgenic animals of the invention can be animals expressing a transgene encoding a target:intein hybrid protein or fragment thereof or variants thereof, including mutants and polymorphic variants thereof. These animals can be used to determine the effect of expression of a target gene protein in a specific site or in a specific temporal window. In one aspect, the invention features a cell or cell line, which contains a knock-in of an intein which has been inserted into a particular target gene. In a preferred embodiment, the cell or cell line is an undifferentiated cell, for example, a stem cell, embryonic stem cell, oocyte or embryonic cell.

Yet in a further aspect, the invention features a method of producing a non-human mammal with a targeted disruption in an interleukin-1 gene. For example, a target gene knock-in construct can be created with a portion of the target gene having an internal portion of said target gene replaced by a marker. The knock-out construct can then be transfected into a population of embryonic stem m(ES) cells. Transfected cells can then be selected as expressing the marker. The transfected ES cells can then be introduced into an embryo of an ancestor of said mammal. The embryo can be allowed to develop to term to produce a chimeric mammal with the knock-out construct in its germline. Breeding said chimeric mammal will produce a heterozygous mammal with a targeted disruption in the target gene. Homozygotes can be generated by crossing heterozygotes.

In another aspect, the invention features target knock-out constructs, which can be used to generate the animals described above. In one embodiment, the target construct can comprise a portion of the target gene, wherein an internal portion of said target gene is replaced by a selectable marker. Preferably, the marker is the neo gene and the portion of the target gene is at least 2.5 kb long or 7.0 or 9.5 kb long (including the replaced portion and any target flanking sequences). The internal portion preferably covers at least a portion of an exon and in some embodiments it covers all of the exons which encode an target polypeptide.

Yet other non-human animals within the scope of the invention include those in which the expression of the endogenous Target gene has been mutated or “knocked out”. A “knock out” animal is one carrying a homozygous or heterozygous deletion of a particular gene or genes. These animals could be useful to determine whether the absence of the target polypeptide will result in a specific phenotype, in particular whether these mice have or are likely to develop a specific disease, such as high susceptibility to heart disease or cancer. Furthermore these animals are useful in screens for drugs which alleviate or attenuate the disease condition resulting from the mutation of the target gene as outlined below. These animals are also useful for determining the effect of a specific amino acid difference, or allelic variation, in a target gene.

In a preferred embodiment of this aspect of the invention, a transgenic target gene knock-in mouse, carrying the mutated target locus on one or both of its chromosomes, is used as a model system for transgenic or drug treatment of the condition resulting from loss of target gene expression.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a specific gene of interest in ES cells, these changes can be introduced into the germlines of animals to generate chimeras. The gene targeting procedure is accomplished by introducing into tissue culture cells a DNA targeting construct that includes a segment homologous to a target locus, and which also includes an intended sequence modification to the target genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a target gene function through the use of a targeting transgene construct designed to undergo homologous recombination with one or more target genomic sequences. The targeting construct can be arranged so that, upon recombination with an element of at gene, a positive selection marker is inserted into (or replaces) coding sequences of the gene. The inserted sequence functionally disrupts the target gene, while also providing a positive selection trait. Exemplary targeting constructs are described in more detail below.

Generally, the embryonic stem cells (ES cells ) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. MoIBRhol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for target or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targetted gene, resulting in a transgenic animal which expresses a polypeptide of the targetted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the target gene knocked out in various tissues of the offspring by probing the Western blot with an antibody against the particular target protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a Target-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A targetted transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a target protein can be essential for many forms of lineage analysis and can additionally provide a means to assess the effects of, for example, lack of target gene expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target proteins. For example, excision of a target sequence which interferes with the expression of a recombinant target gene, such as one which encodes an antagonistic homolog or an antisense transcript, can be designed to activate expression of that gene. This interference with expression of the protein can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant target protein can be regulated via control of recombinase expression.

Use of the cre/loxP recombinase system to regulate expression of a recombinant target protein requires the construction of a transgenic animal containing transgenes encoding both the Cre recombinase and the subject protein. Animals containing both the Cre recombinase and a recombinant target gene can be provided through the construction of “double” transgenic animals. A convenient method for providing such animals is to mate two transgenic animals each containing a transgene, e.g., a target gene and recombinase gene.

One advantage derived from initially constructing transgenic animals containing a target transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic target transgene is silent will allow the study of progeny from that founder in which disruption of target mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the target transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a target gene:intein transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed)

In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a target protein (either agonistic or antagonistic), and antisense transcript, or a target mutant. Further, in such embodiments the sequence will be attached to a transcriptional control element, e.g., a promoter, which preferably allows the expression of the transgene product in a specific type of cell.

Retroviral infection can also be used to introduce transgene into a nonhuman animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

### 4.10. Screening Assays for Intein Signaling Agents

An intein signaling agent can be any type of compound, including a protein, a peptide, peptidomimetic, small molecule, and nucleic acid. A nucleic acid can be, e.g., a gene, an antisense nucleic acid, a ribozyme, or a triplex molecule. An intein signaling agent of the invention can be an agonist or an antagonist. Preferred intein agonists include intein-interacting proteins or derivatives thereof which affect an intein self-excision activity.

The invention also provides screening methods for identifying intein signaling agents which are capable of binding to an intein protein, e.g., a wild-type intein protein or a mutated form of an intein protein, and thereby modulate the self-excision activity of an intein or otherwise prevent the removal of the intein. For example, such an intein modulating agent can be an antibody or derivative thereof which interacts specifically with a wild-type intein protein and thereby antagonizes its self-excision activity. An intein modulating agent may also be a small molecule agonist which binds to a conditional mutant intein polypeptide and thereby activates the conditional mutant by, for example, stabilizing an active form of the conditional intein polypeptide. Thus, the invention provides screening methods for identifying intein agonist and antagonist compounds, comprising selecting compounds which are capable of interacting with an intein protein or with a molecule capable of interacting with an intein protein. In general, a molecule which is capable of interacting with an intein protein is referred to herein as “intein binding partner”.

The compounds of the invention can be identified using various assays depending on the type of compound and activity of the compound that is desired. In addition, as described herein, the test compounds can be further tested in animal models. Set forth below are at least some assays that can be used for identifying intein modulating agents. It is within the skill of the art to design additional assays for identifying intein modulating agents.

### 4.11. Cell-Free Assays

Cell-free assays can be used to identify compounds which are capable of interacting with an intein protein or binding partner, to thereby modify the activity of the intein protein or binding partner. Such a compound can, e.g., modify the structure of an intein protein or binding partner and thereby affect its activity. Cell-free assays can also be used to identify compounds which modulate the interaction between an intein protein and an intein binding partner, such as a target peptide. In a preferred embodiment, cell-free assays for identifying such compounds consist essentially in a reaction mixture containing an intein protein and a test compound or a library of test compounds in the presence or absence of a binding partner. A test compound can be, e.g., a derivative of an intein binding partner, e.g., a biologically inactive target peptide, or a small molecule.

Accordingly, one exemplary screening assay of the present invention includes the steps of contacting an intein protein or functional fragment thereof or an intein binding partner with a test compound or library of test compounds and detecting the formation of complexes. For detection purposes, the molecule can be labeled with a specific marker and the test compound or library of test compounds labeled with a different marker. Interaction of a test compound with an intein protein or fragment thereof or intein binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the intein protein, functional fragment thereof, intein analog or intein binding partner is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) an intein polypeptide, (ii) an intein binding partner, and (iii) a test compound; and (b) detecting interaction of the intein and the intein binding protein. The intein polypeptide and intein binding partner can be produced recombinantly, purified from a source, e.g., plasma, or chemically synthesized, as described herein. A statistically significant change (potentiation or inhibition) in the interaction of the intein and intein binding protein in the presence of the test compound, relative to the interaction in the absence of the test compound, indicates a potential agonist (mimetic or potentiator) or antagonist (inhibitor) of intein self-excision bioactivity for the test compound. The compounds of this assay can be contacted simultaneously. Alternatively, an intein protein can first be contacted with a test compound for an appropriate amount of time, following which the intein binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified intein polypeptide or binding partner is added to a composition containing the intein binding partner or intein polypeptide, and the formation of a complex is quantitated in the absence of the test compound.

Complex formation between an intein protein and an intein binding partner may be detected by a variety of techniques. Modulation of the formation of complexes can be quantitated using, for example, detectably labeled proteins such as radiolabeled, fluorescently labeled, or enzymatically labeled intein proteins or intein binding partners, by immunoassay, or by chromatographic detection.

Typically, it will be desirable to immobilize either the intein or its binding partner to facilitate separation of complexes from uncomplexed forms of one or both of the proteins, as well as to accommodate automation of the assay. Binding of an intein to an intein binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/intein (GST/intein) fusion proteins can be adsorbed onto glutathione sepharose beads (Sigma Chemical, St. Louis, Mo.) or glutathione derivatized microtitre plates, which are then combined with the intein binding partner, e.g. an 35S-labeled intein binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of intein protein or intein binding partner found in the bead fraction quantitated from the gel using standard electrophoretic techniques.

Other techniques for immobilizing proteins on matrices are also available for use in the subject assay. For instance, either the intein or its cognate binding partner can be immobilized utilizing conjugation of biotin and streptavidin. For instance, biotinylated intein molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with an intein can be derivatized to the wells of the plate, and intein trapped in the wells by antibody conjugation. As above, preparations of an intein binding protein and a test compound are incubated in the intein presenting wells of the plate, and the amount of complex trapped in the well can be quantitated. Exemplary methods for detecting such complexes, in addition to those described above for the GST-immobilized complexes, include immunodetection of complexes using antibodies reactive with the intein binding partner, or which are reactive with intein protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the intein binding partner. To illustrate, the intein binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-intein antibodies, can be used. Alternatively, the protein to be detected in the complex can be “epitope tagged” in the form of a fusion protein which includes, in addition to the intein sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which interact with an intein protein and modulate an activity of an intein protein. Accordingly, in one embodiment, an intein protein is contacted with a test compound and the catalytic activity of intein is monitored. In one embodiment, the abililty of the intein to bind a target molecule is determined. The binding affinity of the intein to a target molecule can be determined according to methods known in the art.

### 4.12. Cell Based Assays

The invention further provides certain cell-based assays for the identification of intein modulating agents which agonize or antagonize the self-excision activity of a wild type or conditional mutant intein. In one embodiment, the effect of a test compound on the expression of an intein-containing gene is determined by transfection experiments using a reporter gene comprising a conveniently assayed marker into which has been inserted the subject intein polypeptide sequence. The reporter gene can be any gene encoding a protein which is readily quantifiable, e.g, the luciferase or CAT gene. Such reporter gene are well known in the art. The test compound is contacted with the reporter gene expressing cell line and the amount of reporter (e.g. CAT) activity produced in the presence of a test compound is compared to the amount of activity produced in the absence of the test compound.

In preferred embodiments, the cell-based assays of the present invention make of use of the genetic complementation of a particular biological phenotype by the target:intein polypeptide for the purpose of identifying intein self-excision agonist and antagonist compounds. For example, the complementation of a yeast gal4 mutant phenotype, characterized by an inability to grow on a media containing galactose as the sole carbon source, by a GAL4:intein hybrid protein is dependent upon intein self-excision from the hybrid protein. Screening for intein self-excision agonist and antagonist compounds may thus be effected by contacting the gal4 GAL4:intein yeast strain with a test compound and measuring a galactose growth characteristic in the presence and in the absence of the compound. Suitable galactose growth characteristics include colony size and doubling time on galactose media. An intein self-excision to may be used to identifyt agonist and antagonists which affect this galactose growth phenotype.

Another generally-applicable cell based assays useful for the identification of intein self-excision agonists and antagonists is the yeast two-hybrid assay (Gyuris et al. (1993) Cell 75: 791-803) which is readily adaptable to isolating natural (e.g from a cDNA expression library) or synthetic (detected from a library of random open reading frames) polypeptides which interact with an intein polypeptide of the invention. This intein polypeptide/intein polypeptide binding partner interaction can be further adapted to screens which increase or decrease this intein polypeptide/intein polypeptide binding partner interaction, thereby allowing detection of intein self-excision agonists and antagonists.

### 5. EXAMPLES

### Example 1

### Isolating Conditional Intein Mutants in Yeast

In this example, a Saccharomyces-derived intein was inserted into a derivative of the yeast GAL4 transcriptional activator and the resulting construct was used to obtain cold sensitive and temperature sensitive conditional intein alleles. Thus, a specific polypeptide bioactivity (i.e. GAL 1, 10 transcriptional activation) can be controlled by a signal (such as exposure to low temperature or high temperature) which affects the auto-excision activity of an inactivating intein inserted into the polypeptide encoding that bioactivity.

First, the full length GAL4 coding region was amplified from the plasmid pGaTB (Brand and Perrimon, (1993) Development 118: 401-15) by PCR so as to include a Drosophila translation initiation consensus ATG and a Myc epitope tag at the C terminal end (last 10 amino acids). This product was then subcloned into the pS5DH yeast vector using BamHI and Asp718 at the 5′ and 3′ ends respectively. pS5DH is a centromeric, URA3+ yeast/E. Coli shuttle vector (Gietz and Sugino (1988) Gene 74: 527-34) modified to contain the strong constitutive Adh promoter (Susan Smith unpublished) which has been further modified to remove a HindIII within the polylinker. The resulting construct was then transformed into a URA3- and GAL4-deleted strain of yeast called FY760. Ura+ colonies could grow on galactose containing media whereas Ura+ cells transformed with just the empty vector did not. These manipulations created a yeast Adh:GAL4* centromeric expression vector capable of supporting growth on media in which galactose is the sole carbon source.

This Adh:GAL4* construct was then modified so that the sequence from position 54 to 65 was AAA AAG CTT AAG. This added a unique HindIII site (AAGCTT) and destroyed an existing AflII site. In addition a new silent AflII site was added into Gal4 (position 1461 to 1466 in the final sequence). This modified Gal4 construct was tested once more for its ability to rescue FY760 for growth on media in which galactose is the sole carbon source and is known as pS5-Gal4.

Next, the INTEIN within the S. cerevisiae VMA1 gene was amplified by PCR from genomic yeast DNA, and was subsequently subcloned into pBS (Stratagene) and sequenced. An internal HindIII restriction site within the INTEIN was destroyed by PCR based in vitro mutagenesis. This construct was then amplified by PCR primers that included the Gal4 sequence AAG CTT AAA at the 5′ end and the Gal4 derived sequence TCC AAA GAA AAA CCG AAG TGC CCA AGT GTC TTA AG at the 3′ end. With the HindIII and AflII restriction sites added to the end of the INTEIN sequence this product was subcloned into the modified pS5-Gal4 gapped with HindIII and AflII. The resulting pS5-Gal4INT construct was also tested for its ability to rescue FY760 and found to enable growth as efficiently as pS5-Gal4 lacking the INTEIN. Thus, these procedures resulted in the production of a yeast centromeric expression vector capable of expressing a GAL4*::INTEIN hybrid protein which could functionally complement a gal4 mutation.

An alternative approach to inserting the INTEIN nucleic acid sequence into the target polypeptide-encoding sequence is to perform this operation in vivo in yeast In this alternative method the INTEIN would be PCR amplified by long primers that include at least about 60 bp of sequence homologous to the target region within Gal4 on either side of the desired INTEIN integration site. This PCR product is then co-transformed into FY760 yeast together with the pS5-Gal4 plasmid which has been linearized by a restriction site situated close to the desired insertion site. As linear plasmids do not replicate in yeast, only molecules in which homologous recombination between the plasmid and the two ends of the PCR fragment has taken place will result in a circularized, viable plasmid containing the INTEIN.

Finally, temperature sensitive and cold sensitive derivatives of this GAL4*::INTEIN hybrid protein-producing vector were isolated. The INTEIN sequence within pS5-Gal4INT was used as a template for mutagenic low fidelity PCR using primers just outside the unique HindIII and AflII sites. The resulting product was trimmed and subcloned into gapped pS5-Gal4. The resulting ligation was transformed into ultra-competent E. coli cells and grown up in liquid culture as an amplification step. DNA extracted from this culture was used to transform FY760 yeast before plating onto URA-selective dextrose plates. The colonies that grew on these plates were then replica plated onto two URA-selective galactose plates which were grown at 18 and 30 [symbol] C. Colonies that grew at different rates on these two plates were identified and re-tested for temperature sensitivity and the plasmids they contained were recovered. These plasmids were then re-transformed into FY760 to ensure that the TS phenotype was plasmid related, the INTEIN within the pS5-Gal4INT molecules was sequenced.

### Example 2

### Use of TS Conditional Intein Mutants to Control Other Proteins

In order to confirm that the INTEIN TS alleles already generated in a Gal4 context are autonomously TS (ie. host context independent) we have moved the two alleles (TS1 and TS18) into Gal80 (a negative regulator of Gal4). The resulting Gal80INT constructs are then constitutively expressed in wild type yeast and growth on a galactose carbon source is assessed. If functional Gal80 is produced, endogenous Gal4 is down regulated and no growth results. If the presence of the INTEIN in Gal80 disrupts the protein function then endogenous Gal4 is not affected and cells will grow normally.

A total of 4 positions were analyzed (immediately upstream of C127, S193, C277 and T299). Using the wild type (WT) INTEIN and a ‘dead’ INTEIN previously shown not to splice (see Gal4 report above) we established that the VMA1 INTEIN must be positioned upstream of a Cystine residue (ie. at C127 or C277). Other INTEINS have been described as being present upstream of Serine and Threonine aminoacids hence the attempt to use these residues in this case.

The WT and dead intein controls acted as would be expected—i.e. the Gal80::INTEIN^{WT} construct was capable of repressing growth on galactose while the Gal80::INTEIN^{DEAD} construct was not capable of repressing growth on galactose. Interestingly, when the conditional intein alleles were inserted upstream of Gal80 C277, they conferred different phenotypes upon the mutant gal80 protein, implying that they established different levels of steady-state wild type spliced protein. The TS1 and TS18 mutant inteins, when inserted at C127 of Gal80, did not significantly interfere with growth on galactose, implying that relatively low levels of spliced Gal80 protein resulted. These two alleles appear not to splice and growth is essentially the same as for the Gal80INT-dead construct. In contrast, the two TS alleles, when inserted at C227, inhibited growth on galactose at both the permissive temperature (i.e. 18 [symbol] C) and the restrictive temperature (i.e. 30 [symbol] C), implying that relatively large amounts of spliced wild-type Gal80 protein are produced even at the restrictive temperature. These results suggest that, depending upon the protein context into which the conditional intein is inserted, different levels of spliced versus unspliced protein can be achieved. These results will be confirmed by the analysis of gross levels of spliced and unspliced Gal80 protein using an immunoprecipitation and Western blotting assay.

Therefore the invention is adaptable to the regulation of active protein concentrations at various levels depending upon the site of insertion into the target protein.

We are still further pursuing two other lines of investigation to generate still other working examples. The first is to move the other available TS alleles into the two C127 and C277 positions in an attempt to identify one of the alleles as being strictly autonomously TS for the galactose growth phenotype when placed in the context of Gal80.

Another approach we are taking is to move the TS INTEINS together with a small region of the context in which they were generated (in Gal4). It has been shown that the INTEIN interacts with residues of the host protein immediately up and downstream of its insertion site during splicing (see Nogami et al. (1997) Genetics 147:73). Therefore it is possible that the galactose phenotype of the TS alleles tested in Gal80 may be due to the temperature sensitive nature of the interactions of the INTEIN with these flanking amino acids. Thus the transfer of these residues together with the INTEIN may maintain the conditional nature of the system.

We will also insert the TS1 and TS18 INTEINS into GFP together with a short region (2-4 amino acids) flanking the original insertions. By using the commercially available anti-GFP antibodies and PAGE/Western blot analysis we will test to see if this then results in host protein “independent” splicing. Obviously this approach would result in a short stretch of “foreign” amino-acids being left in the host protein but may represent one approach with which the system could be optimized.

We further note here that if an autonomously acting TS alleles is identified it may be possible to ‘improve’ its characteristics by further rounds of mutagenesis (as was accomplished, for example, in some of the screens for brighter GFP molecules).

Still further, we note that if the “flanking” ‘pieces are required to make a conditional system it may be possible to utilize this sequence for particular purposes. For example, these flanks will only come together after splicing and could potentially be used as a tag (given the production of suitable antibodies) with which to identify functional (spliced) host protein. These tagged intein constructs could be utilized in screens to identify interacting compositions which agonize or antagonize the intein splicing reaction.

### Example 3

### Use of Condition-Sensitive Mutants in Plants

Low temperature is a major environmental limitation to the production of agricultural crops. For example, late spring frosts delay seed germination, early fall frosts decrease the quality and yield of harvests and winter low temperatures decrease the survival of overwintering crops, such as winter cereals and fruit trees. However, some plants have the ability to withstand prolonged subfreezing temperatures. If proteins involved in the development of frost tolerance in these plants, as well as the corresponding genes, can be identified, it may be possible to transform frost sensitive crop plants into frost tolerant crop plants and extend the range of crop production.

Biological organisms can survive icy environments by inhibiting internal ice formation. This strategy requires the synthesis of antifreeze proteins (AFPs) or thermal hysteresis proteins (THPs). Four distinct types of (AFPs) have been identified in fish and a number of different THPs have been identified in insects. These previous findings suggest that this adaptive mechanism has arisen independently in different organisms. Antifreeze proteins are thought to bind to ice crystals to prevent further growth of the crystals. The presence of antifreeze proteins can be determined (1) by examining the shape of ice crystals as they form and (2) by measuring the existence of thermal hysteresis (the difference in temperature at which a particular solution melts and freezes).

It was generally understood that antifreeze proteins did not exist in plants. Instead, it was thought that some internal mechanism of the plant cells adapted them to withstand external ice crystal formation on their outer cell walls without damaging the cell. For example, a plant gene expressed at low temperature codes for a protein similar in amino acid sequence to the antifreeze protein, did not have sufficient amounts of the encoded protein to determine whether it exhibited an antifreeze activity in the plant and particularly within the plant cell. Fish antifreeze protein to can increase frost tolerance in plants.

Examples of plant anti-freeze include the Arachis hypogaea cold shock protein (AHCSP33), Dave et al. (1998) Phytochemistry 49:2207-13; a carrot leucine-rich-repeat-protein that inhibits ice re-crystallization, which is similar to the anti-freeze proteins found in fish and which accumulates antifreeze activity when expressed in transgenic tobacco plants, (Worrall et al., (1998) Science 282:115-117); an arabidopsis thaliana cold induced kin1 gene, a alanine, glycine, and lysine-rich protein, which protein is also induced by osmotic stress (Kurkela et al. (1990) Plant Mol. Biol. 15:137-144); (Tahtiharju et al (1997) Planta 203:442-447); antifreeze proteins in rye are reported as being similar to pathogenesis-related proteins such as endochitinases (Hon et al. Plant Physiol. 91995) 109(3):879-89. Furhermore other studies of cold-inducibe genes in plants have suggested the existence of family of cold-resistant polypeptides. A rapid and stable change occurs in the translatable poly(A).sup.+RNA populations extracted from leaves of plants exposed to low temperatures. Total protein analysis of the plant tissues was conducted to detect proteins which might be associated with frost tolerance in plants. Proteins found in cold acclimated leaf extracts having molecular weights of 110 kd, 82 kD, 66 kD, 55 kD and 13 kD were not found in non-acclimated leaf extracts. It is thought that the increased expression of certain mRNAs may encode proteins that are involved directly in a development of increased freezing tolerance for the plant. High molecular mass proteins which are believed to be associated with cold acclimation in spinach. The total protein content of the acclimated spinach leaf is assessed. Cold acclimated proteins having molecular weights of 110 kD, 90 kD and 79 kD were identified. However, their location and function within the cell remain unknown.

In certain instances cold tolerance has been conferred by transgenic expression of for e.g., a synthetic anti-freeze protein in potato plants (Wallis et al. (1997) Plant Mol. Biol., 35:323-330; or a fusion of Staphylococcal protein A and antifreeeze protein (AFP) from polar fish (Hightower et al. (1991) Plant Mol. Biol. 17:1013-1021). Further, certain studies have suggested that accumulation of antifreeze proteins is temperature or cold specific. For instance, constitutive expression of a fish antifreeze protein encoding gene does not lead to measureable antifreeze protein until the plant is exposed to colder conditions, suggesting that such AFP may be inherently unstable at warmer temperatures (Kenward et al 91993) Plant Mol. Biol. 23:377-385).

Therefore in one embodiment, this invention contemplates the constitutive expression of AFP wherein the activity of the AFP polypeptide so expressed may be rapidly induced so as to confer immediate cold tolerance and/or ice crystal growth inhibition in the absence of de novo synthesis. It is known that AFP polypetides depress the freezing temperature of a solution in a non-colligative manner (Chapski et al. 91997) FEBS Let. 412: 241-244). Therefore, the rapid induction of an existing latent cold tolerance bioactivity would be expected to confer superior resistance to sudden frost conditions than mechanisms requiring de novo synthesis of the AFP polypeptides.

Accordingly, in one aspect, this invention contemplates, regulatable AFP proteins comprising condition-sensitive mutant intein, such as AFP proteins comprising mutant temperature sensitive inteins, such as temperature sensitive alleles of S. Cerviseaea vacuolar ATPase catalytic subunit (VMA) intein containing gene. Examples of these temperature sensitive alleles of the Sce. VMA intein sequences are set forth in SEQ ID Nos. 2 to 9 The amino acid changes in the TS alleles due to these specific mutations are listed in Table 3 above, wherein L212P refers to a Leucine→Proline change at position 212.

In one example, a temperature sensitive allele is inserted into an AFP gene from winter flounder which codes for an alanine-rich alpha helical type I AFP. Plants may be transformed with an expression vector comprising the AFP-intein hybrid. Transformation may be accomplished by any of the methods which have been well documented in the art.

In particular, various methods are known to one of ordinary skill in the art to accomplish such genetic transformation of plants and plant tissues. For example, these methods include transformation by Agrobacterium species and transformation by direct gene transfer. These method are described in detail in U.S. Pat. No. 5,789,214, which is incorporated herein by reference.

The Agrobacterium system permits routine transformation of a variety of plant tissue, examples of such plants include tobacco, tomato, sunflower, cotton, rapeseed, potato, soybean, and poplar. While the host range for Ti plasmid transformation using A. tumefaciens as the infecting agent is known to be very large, tobacco has been a host of choice in laboratory experiments because of its ease of manipulation. Another example is Agrobacterium rhizogenes which has also been used as a vector for plant transformation. Transformation using A. rhizogenes has been successfully utilized to transform, for example, alfalfa, Solanum nigrum L., and poplar.

In addition, the art also discloses many direct gene transfer procedures which have been developed to successfully transform plants transform plants and plant tissues without the use of an Agrobacterium intermediate (see, for example, Koziel et al., Biotechnology 11: 194-200 (1993). For example, exogenous DNA can be introduced into cells or protoplasts by microinjection. (Reich, T. J. et al., Bio/Technology 4: 1001 (1986). Another example involves bombardment of cells by microprojectiles carrying DNA, see Klein, T. M. et al., Nature 327: 70 (1987).

Accordingly, tobacco plants may be transformed using any of the methods described above, with an AFDP-intein gene consruct which is expressed from the Cauliflower Mosaic virus 19S RNA promoter using Nopaline synthetase polyadenylation site. Expression of the AFP-intein may be confirmed by Western blot analysis. Accumulation of (non-functional) AFP was observed at warmer temperatures, and it was observed that a shift to colder temperatures results in the formation of functional AFP and an excised autonomous intein.

### Example 4

### Inducibly Trans-Spliced Thymidine Kinase

In a second example, an intein trans-spliced regulatable form of thymidine kinase is constructed and expressed under the control of a pituitary hormone promoter (human GH or glycoprotein hormone alpha-subunit) using recombinant adenoviral vectors. Injection into nude mice carrying propagated GH3 cell pituitary adenomas results in gancyclovir-dependent cytotoxicity which is further dependent upon a chemical signal (rapamycin) to trigger trans-splicing of the thymidine kinase exteins into a single mature thymidine kinase polypeptide. The added level of control provided by the rapamycin chemical signal affords greater flexibility in achieving optimal tumor cell cytotoxicity in a temporally regulatable manner. Further advantages include regulating drug toxicity and assuring cell specificity in the host organism.

First, in order to ensure that the insertion of the regulatably trans-spliced intein disrupts the thymidine kinase bioactivity of the target polypeptide, a BLAST protein alignment with the target human herpes simplex virus thymidine kinase polypeptide sequence is performed. Two representative matches with related viral thymidine kinase genes from other host species are shown below. This step assures that the trans-spliced intervening protein sequence segments are appropriately inserted so as to interfere with the target protein's activity. Covalent separation of two major segments of a target polypeptide and concomitant fusion of the end of these segments to intervening protein sequences is unlikely to fail to disrupt the target polypeptide's bioactivity. Nonetheless, this step ensures that the trans-spliced intein units are not placed so as to disrupt an unconserved, nonessential amino- or carboxy-terminal portion of the polypeptide. Furthermore, such an analysis assures that the site of the disrupting trans-spliced intein does not correspond to an unconserved “linker” sequence, without which the amino and carboxy exteins might still reassemble by virtue of inherent protein domain/protein domain affinities. Indeed in The BLAST homology searching program (NCBI's sequence similarity search tool) was used to identify homologs of the Herpes Simplex Virus type 2 thymidine kinase (TK) polypeptide sequence (Swiss-Prot. Acc. No. 3915741) to be used in the experiment. Representative related viral TK polypeptide sequences are shown below. Comparison the human type 2 TK sequence (Query) to both a bovine HSV viral TK homolog (TK homolog 1, Subject) and a related pseudorabies viral TK homolog (TK homolog 2, Subject) reveals several candidate conserved serine (S), threonine (T) and cysteine (C) residues which are conserved in both evolutionarily distant homologs. The cysteine at amino acid 172 of the human HSV TK polypeptide is chosen on the basis of: it's chemical suitability for intein excision as an amino terminal end of a carboxy-extein; it's presence near the center of the polypeptide, flanked by regions of conserved sequence; and it's presence in a large block of strictly conserved sequence, contraindicative of a dispensable polypeptide loop domain.

Whereas for most polypeptides specific guidance for insertion site selection will be easily obtained by comparison with other proteins with the same bioactivity, in certain instances, such as the instant example, additional guidance will be available in the form of protein crystal structure studies (see e.g. http://www.ncbi.nlm.nih.gov/Structure/which provides access to a large bank of proteins for which crystal structures are available).

| TK homolog 1 (from bovine HSV; Swiss-Prot. Acc. No. 125440) | | | |
|---|---|---|---|
| Query: | 49 | LLRVYIDGPHGVGKTTTSAQLMEALGPRDNIVYVPEPMTYWQVLGASETLTNIYNTQHRL | 108 |
| | | LLRVY+DGPHG+GKTT+++L G ++Y+PEPM+YW G ++ +Y QHR+ | |
| Sbjct: | 4 | LLRVYVDGPHGLGKTTAASRLASERG---DAIYLPEPMSYWSGAGEDDLVARVYTAQHRM | 60 |
| Query: | 109 | DRGEISAGEAAVVMTSAQITMSTPYAATDAVLAPHIGGEAVGPQAPPPALTLVFDRHPIA | 168 |
| | | DRGEI A EAA V+ AQ+TMSTPY A + ++A PP L L+FDRHP A | |
| Sbjct: | 61 | DRGEIDAREAAGVVLGAQLTMSTPYVALNGLIAPHIGEEPSPGNATPPDLILIFDRHPTA | 120 |
| Query: | 169 | SLLCYPAARYLMGSMTPQAVLAFVALMPPTAPGTNLVLGVLPEAEHADRLARRQRPGERL | 228 |
| | | SLLCYP ARYL + ++VL+ +AL+PPT PGTNL+LG P +H RL R PGE | |
| Sbjct: | 121 | SLLCYPLARYLTRCLPIESVLSLIALIPPTPPGTNLILGTAPAEDHLSRLVARGPPGELP | 180 |
| Query: | 229 | DLAMLSAIRRVYDLLANTVRYLQRGGRWREDWGRLTGVAAATPRPDPEDGAGSLPRIEDT | 288 |
| | | D ML AIR VY LLANTV+YLQ GG WR D G P PEG +P +T | |
| Sbjct: | 181 | DARMLRAIRYVYALLANTVKYLQSGGSWRADLG---SEPPRLPLAPPEIGDPNNPGGHNT | 237 |
| Query: | 289 | LALFRVPELLAPNGDLYHIFAWVLDVLADRLLPMHLF 325 | |
| | | LL +A G ++W LD+LADRL M++F | |
| Sbjct: | 238 | L-LALIHGAGATRG-CAAMTSWTLDLLADRLRSMNMF 272 | |

| TK homolog 2 (from Pseudorabies virus (STRAIN NIA-3); Swiss-Prot. Acc. No.125456) | | | |
|---|---|---|---|
| Query: | 49 | LLRVYIDGPHGVGKTTTSAQLMEALGPRDNIVYVPEPMTYWQVLGASETLTNIYNTQHRL | 108 |
| | | +LR+Y+DG+ GK+TT+ + ALG +YVPEPM YW+L ++T+IY+ Q R | |
| Sbjct: | 3 | ILRIYLDGAYDTGKSTTARVM--ALG---GALYVPEPMAYWRTLFDTDTVAGIYDAQTRK | 57 |
| Query: | 109 | DRGEISAGEAAVVMTSAQITMSTPYAATDAVLAPHIGGEAVGPQAPPPALTLVFDRHPIA | 168 |
| | | G+S +AA+V Q +TPY LP G GP P+T+VFDRHP+A | |
| Sbjct: | 58 | QNGSLSEEDAALVTAHDQAAFATPYLLLHTRLVPLFGPAVEGP----PEMTVVFDRHPVA | 113 |
| Query: | 169 | SLLCYPAARYLMGSMTPQAVLAFVALMPPTAPGTNLVLGVLPEAEHADRLARRQRPGERL | 228 |
| | | + +C+P AR+++G++ A+A+P PG NLV+ L EH RL R R GE+ | |
| Sbjct: | 114 | ATVCFPLARFIVGDISAAAFVGLAATLPGEPPGGNLVVASLDPDEHLRRLRARARAGEHV | 173 |
| Query: | 229 | DLAMLSAIRRVYDLLANTVRYLQRGGRWREDWGRLTGVAAAT-----------PRPDPED | 277 |
| | | D +L+A+R VY +L NT RYL G RWR+DWGR T PR DPE | |
| Sbjct: | 174 | DARLLTALRNVYAMLVNTSRYLSSGRRWRDDWGRAPRFDQTTRDCLALNELCRPRDDPE- | 232 |
| Query: | 278 | GAGSLPRIEDTL-ALFRVPELLAPNGDLYHIFAWVLDVLADRLLPMHLFVLDYDQSPVGC | 336 |
| | | ++DTL ++ PEL G +AW +D L +LLP+ + +D SP C | |
| Sbjct: | 233 | -------LQDTLFGAYKAPELCDRRGRPLEVHAWAMDALVAKLLPLRVSTVDLGPSPRVC | 285 |
| Query: | 337 | RDALLRLTAGMIPTRVTTAGSIAEIRDLARTFAREVG 373 | |
| | | A+ TGM VT+ IR F E+G | |
| Sbjct: | 286 | AAAVAAQTRGM---EVTESAYGDHIRQCVCAFTSEMG 319 | |

Therefore an appropriate set of constructs for creating the trans-spliced TK polypeptide would be: TK_{codons1-171}-INTEIN^{N} and INTEIN^{C}-TK_{codons172(cys)-376}. These two polypeptides are modified further so as to subject them to regulated trans-transplicing as described below.

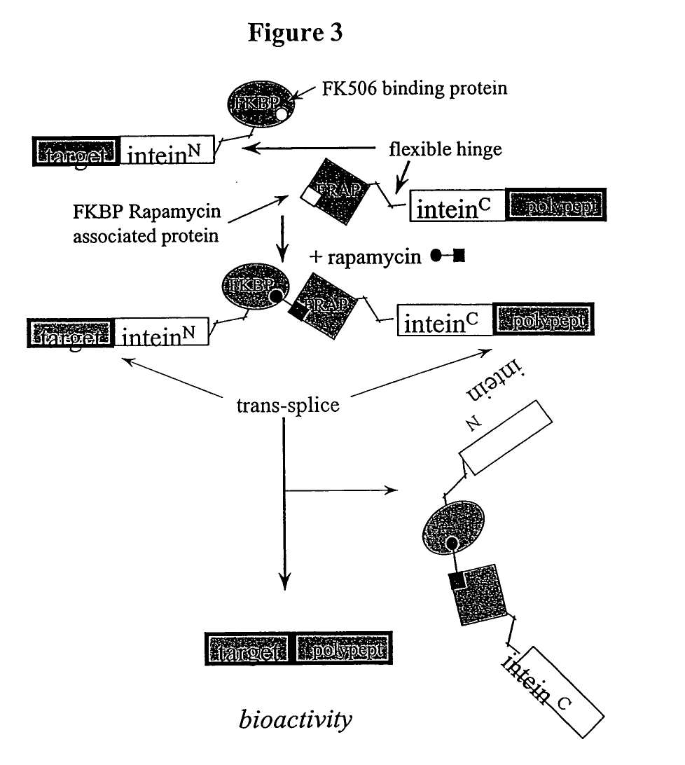

As the instant application is in a mammalian system, the temperature sensitive conditional intein mutants are not readily exploitable. Instead, this example takes advantage of the observation that trans-splicing of an Extein^{N}-Intein^{N} polypeptide to an Intein^{C}-Extein^{C} polypeptide can occur in vitro (Southworth et al. (1998) EMBO J 17: 918-26). The application of inducible trans-splicing to regulation of a hypothetical target polypeptide is diagramed in FIG. 3. Formation of the intein splicing active site requires proper folding of the intein to bring together the two splice junctions, which can be separated by as much as 500 amino acids or more. The in vitro formation of the intein splicing active site was guided by Intein^{N}/Intein^{C} protein/protein interactions. In particular, the Intein^{N} and Intein^{C} sequences collectively comprised the entire Psp Pol-1 Intein-encoded endonuclease which, when proteolytically cleaved into two pieces, is able to reassemble by virtue of “innate” protein/protein affinities (Southworth et al. (1998) EMBO J 17: 918-26). Following noncovalent in vitro association of the Extein^{N}-Intein^{N} and Intein^{C}-Extein^{C} polypeptides, activation of the intein auto-excision function followed spontaneously to yield covalently joined Extein^{N}-Extein^{C} product and a noncovalently joined Intein^{N}:Intein^{C} complex. This in vivo trans-splicing application is expected to function with relative efficiency—indeed certain protein/protein reconstitution have been shown to occur more efficiently in vivo than in vitro (Gross et al. (1996) Protein Sci 5: 320-30). Thus trans-splicing of intein amino and carboxy-terminal domains can occur spontaneously in vitro provided that the intein units are brought together by appropriate intermolecular attractions.

The instant example takes advantage of this observation by using a recently developed chemical dimerizer system (Pruschy et al. (1994) Curr Biol 1: 163-72) to bring the Extein^{N}-Intein^{N} and Intein^{C}-Extein^{C} polypeptides together in a regulatable manner so as to potentiate trans-splicing of the extein units to yield an Extein^{N}-Extein^{C} product.

The chemical dimerizer utilized in this application is capable of crosslinking FKBP (FK506 binding protein) and FKBP Rapamycin Associated Protein (FRAP). FKBP12 belongs to a class of immunophilin proteins, originally discovered because of their high affinity for immunosuppressive drugs. FKBP12 binds to the natural products FK506 and rapamycin with high affinity (K_{D}=0.4 nM and 0.2 nM respectively). The protein has intrinsic peptidyl-prolyl cis-trans isomerase activity, which is blocked on binding to either FK506 or rapamycin, but which does not appear to be related to the ability of these molecules to inhibit intracellular signaling pathways. Instead, their actions are mediated by the formation of composite surfaces in the FKBP12-FK506 and FKBP12-rapamycin complexes that allow binding to calcineurin and the lipid kinase, FKBP-rapamycin-associated protein (FRAP) respectively. Inhibiting the function of calcineurin and FRAP results in the inhibition of different signaling pathways. Studies of FK506 reveal that it possesses two protein-binding surfaces, an immunophilin-binding surface and a calcineurin-binding one; it can thus be termed a “chemical inducer of dimerization” (CID). Two factors that are important in the selection of FK506 as a building block for a designed CIP is its ability to cross cell membranes and its high affinity for FKBPs. To construct an FK506 dimer, two FK506 monomers can be dimerized via a functional group within the calcineurin-binding domain. The resulting dimer still binds to FKBP12, but the complex of the dimer with FKBP12 should not bind to calcineurin and thus should not block TCR signaling. Furthermore, modified chemical dimerizers which bind only to genetically modified forms of FKBP binding proteins are also available and potentially eliminate concerns about undesirable immunosuppressive effects from binding to endogenous FKBP (Clackson et al. (1998) PNAS 95: 10437-42).

In this example, the Extein^{N}-Intein^{N} polypeptide is fused to FKBP and the Intein^{C}-Extein^{C} polypeptide is fused to FRAP. Both FKBP and FRAP are capable of binding simultaneously to rapamycin. In practice either rapamycin binding protein can be used with either amino or carboxy-terminal target polypeptide. A homopolymeric “hinge” region (e.g. polyglycine—polyG) is also added between each target polypeptide fragment and its rapamycin binding protein domain. Such hinge regions are predicted to lack secondary structure following protein folding. As a result, the intein amino and carboxy terminal domains are expected to be free to associate upon dimerization of the FKBP and FRAP domains with rapamycin. The resulting two polypeptides-TK_{codons1-171}-Intein^{N}-polyG-FKBP and FRAP-polyG-Intein^{C}-TK_{codons172(cys)-376} can be stably co-expressed. The thymidine kinase bioactivity can then be induced at any time by delivery of the dimerizer drug rapamycin which causes the non-covalent association of the two protein halves to form TK_{codons1-171} Intein^{N}-polyG-FKBP:rapamycin:FRAP-polyG-Intein^{C}-TK_{codons172(cys)376}. This complex undergoes intein trans-splicing via assocation of the Intein^{N} and Intein^{C} domains, to generate a TK₁₋₃₇₆ complete thymidine kinase polypeptide product and Intein^{N}-polyG-FKBP:rapamycin:FRAP-polyG-Intein^{C} byproduct polypeptide.

The two trans-spliced polypeptide-encoding gene constructs can be delivered to a target cell or tissue by a virus or other suitable delivery system known in the art.

### Equivalents

Those skilled in the art will recognize, or be able to ascertain using no more than routine experimentation, many equivalents of the specific embodiments of the invention described herein. Such equivalents are intended to be encompassed by the following claims.

We claim:

1. A method of increasing or decreasing a bioactivity of a target polypeptide comprising:
inserting an intein into the target polypeptide, wherein said intein is capable of self-excision; and
providing a signal that agonizes or antagonizes the intein excision activity;
thereby increasing or decreasing the bioactivity of the target polypeptide by agonizing or antagonizing the intein excision activity.

2. The method of claim 1, wherein said intein is a conditional mutant intein.

3. The method of claim 1, wherein said conditional mutant intein is a temperature-sensitive intein.

4. The method of claim 3, wherein said intein has reduced self-excision activity at temperatures over about 29° C. relative to its self-excision activity at 18° C.

5. The method of claim 2, wherein said conditional mutant is a cold-sensitive mutant.

6. The method of claim 5, wherein said intein has reduced self-excision activity at temperatures below about 18° C. relative to its self-excision activity at 30° C.

7. The method of claim 1, wherein the signal is selected from the group consisting of changes in temperature, alteration of pH, electromagnetic radiation, phorphorylation or dephosphorylation, glycosylation or deglycosylation, changes in the concentration of an ion, changes in the concentration of a metal ion, changes in osmotic pressure, and addition or inactivation of a chemical ligand.

8. The method of claim 7, wherein the change in temperature is an increase in temperature.

9. The method of claim 7, wherein the change in temperature is a decrease in temperature.

10. The method of claim 7, wherein the chemical ligand is a chemical dimerizer.

11. A method of claim 10, wherein the chemical dimerizer is selected from the group consisting of rapamycin, rapamycin analogs, salicyclic acid and abssicic acid.

12. A method of modulating a bioactivity of a target polypeptide by agonizing or antagonizing the excision of a regulatable intein inserted into the target polypeptide comprising:
providing a regulatable intein, wherein said regulatable intein encodes an intein excision activity that can be agonized or antagonized in response to a signal;
inserting the intein into the target polypeptide which encodes a bioactivity, such that the inserted intein sequence decreases the bioactivity; and
providing a signal that agonizes or antagonizes the intein excision activity;
thereby increasing or decreasing, respectively, the bioactivity of the target polypeptide.

13. The method of claim 12, wherein the regulatable intein is encoded by a nucleic acid that hybridizes under stringent conditions to a nucleic acid selected from the group consisting of SEQ ID Nos. 13, 15, 17 or 19.

14. The method of claim 12, wherein the regulatable intein is encoded by a nucleic acid which is at least 75% identical to the intein-encoding nucleic acid from any of SEQ ID Nos. 13, 15, 17 or 19.

15. The method of claim 12, wherein the regulatable intein has a polypeptide sequence at least 75% homologous to the intein polypeptide sequence of any of SEQ ID Nos. 14, 16, 18 or 20.

16. The method of claim 1 or claim 12, wherein the intein has a polypeptide sequence specified by any of SEQ ID Nos. 2-12.

17. The method of claim 1 or 12, wherein the target polypeptide is GAL4.

18. The method of claim 17, wherein the GAL4 target polypeptide is encoded by a nucleic acid which hybridizes under stringent conditions to the nucleic acid of SEQ ID No. 21.

19. A regulatable intein polypeptide with an amino acid sequence which comprises at least one of the amino acid changes found in a conditional intein allele selected from the group consisting of TS1, TS4, TS8, TS10, TS15, TS17, TS18, TS19, CS1, CS2 and CS3.

20. The regulatable intein polypeptide of claim 19 which has an amino acid sequence of any of SEQ ID Nos. 2-12.

21. A mutant intein polypeptide comprising a block C domain mutation wherein the second residue of said block C domain is mutated to a nonhydrophobic amino acid residue.

22. The mutant intein polypeptide of claim 21, wherein the nonhydrophobic amino acid residue is proline.

23. A mutant intein polypeptide comprising a block E domain mutation wherein the seventh residue of said block E domain is mutated to a nonacidic amino acid residue.

24. The mutant intein polypeptide of claim 23, wherein the nonacidic amino acid residue is glycine.

25. A regulatable intein which is trans-spliced.

26. The regulatable intein of claim 25, comprising an amino-terminal intein polypeptide, a linker polypeptide, a dimerizable domain and a carboxy-terminal intein polypeptide.

27. The regulatable intein of claim 26, wherein said linker polypeptide is selected from the group consisting of Asn-Gly repeats, a polyglycine linker, and Gly-Ser repeats.

28. An isolated nucleic acid which encodes the regulatable intein of any of claims 19, 20, 21, 22, 23, 24, 25, 26 or 27.

29. A regulatable intein polypeptide which is encoded by a nucleic acid that hybridizes under stringent conditions to a nucleic acid selected from the group consisting of SEQ ID Nos. 13, 15, 17 and 19, wherein said intein is a conditional mutant.

30. The regulatable intein of claim 29, comprising a block EN1 domain mutation wherein the second residue of said block EN1 domain is mutated to a nonhydrophobic amino acid residue.

31. The regulatable intein of claim 30, wherein the nonhydrophobic amino acid residue is proline.

32. The regulatable intein of claim 29, comprising a block EN3 domain mutation wherein the seventh residue of said block EN3 domain is mutated to a nonacidic amino acid residue.

33. A regulatable intein of claim 32, wherein the nonacidic amino acid residue is glycine.

34. A regulatable chimeric polypeptide comprising:
a target polypeptide having a bioactivity; and
an intein, which undergoes self-excision, inserted into the target polypeptide, wherein providing a signal that agonizes or antagonizes the intein self-excision activity causes an increase or decrease, respectively, in the bioactivity of the target polypeptide.

35. A regulatable chimeric polypeptide comprising:
a target polypeptide having a bioactivity; and
an intein, which undergoes self-excision, inserted into the target polypeptide, wherein providing a signal that agonizes or antagonizes the intein self-excision activity causes a decrease or increase, respectively, in the bioactivity of the target polypeptide.

36. A nucleic acid encoding the polypeptide of claim 34 or 35.

37. The nucleic acid of claim 34 or 35 wherein the nucleic acid encoding the regulatable chimeric polypeptide is operably linked to a transcriptional regulatory sequence.

38. The nucleic acid of claim 37, wherein the transcriptional regulatory sequence regulates gene expression in mammalian cells.

39. The nucleic acid of claim 36, wherein the regulatable chimeric polypeptide is a GAL4:Intein hybrid polypeptide.

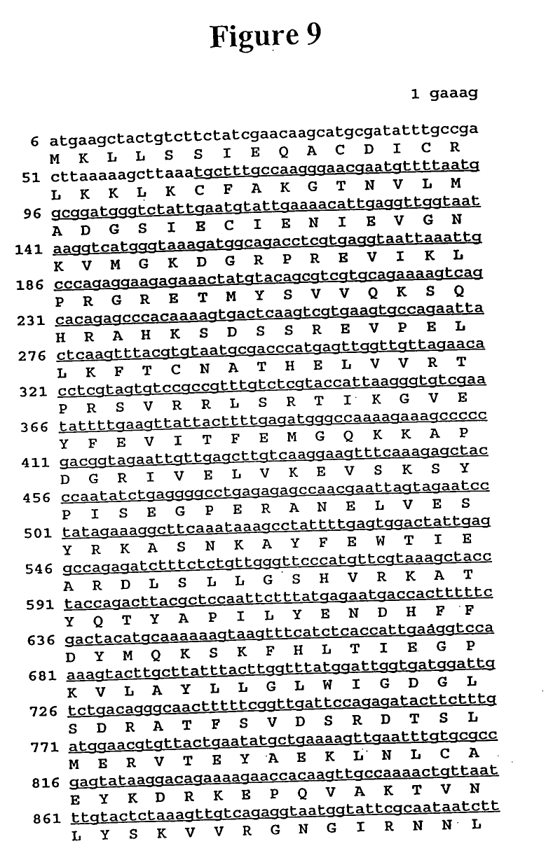

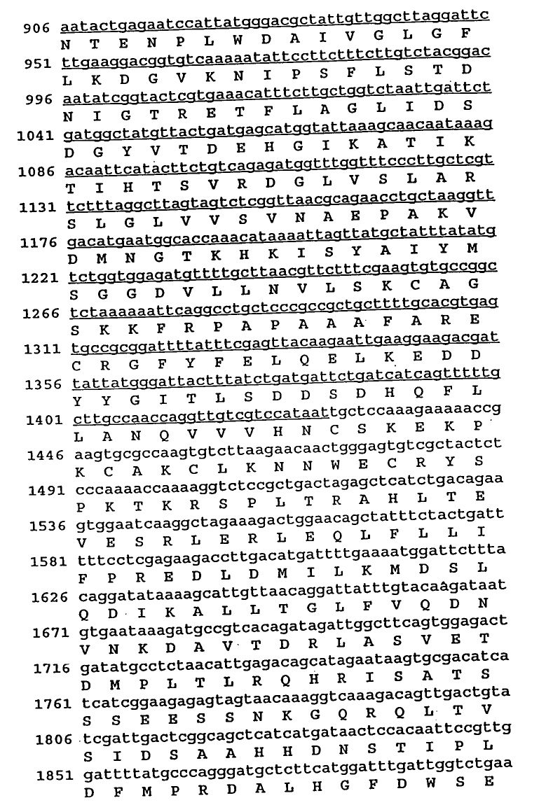

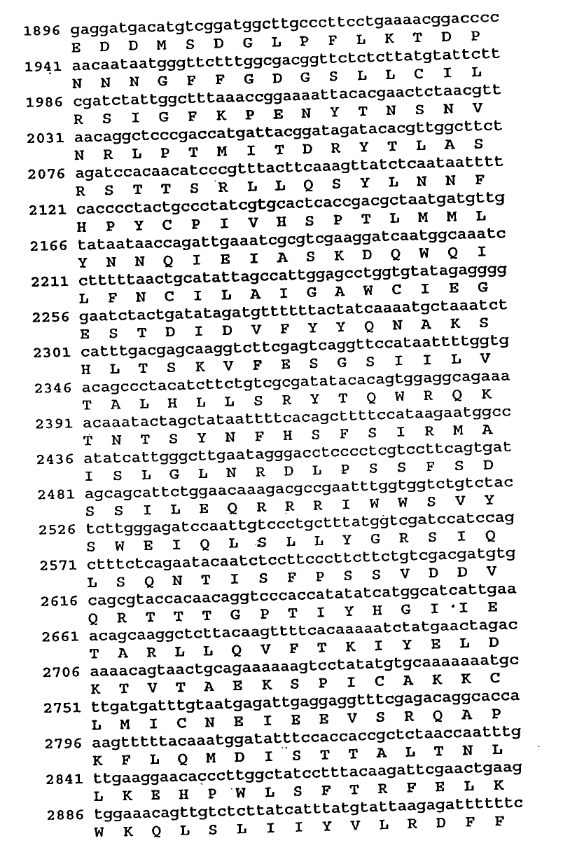

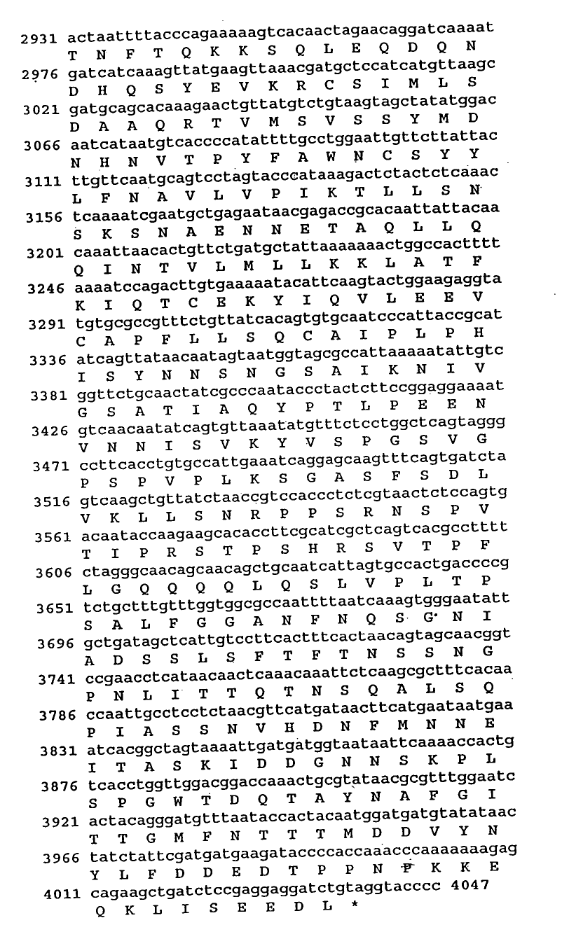

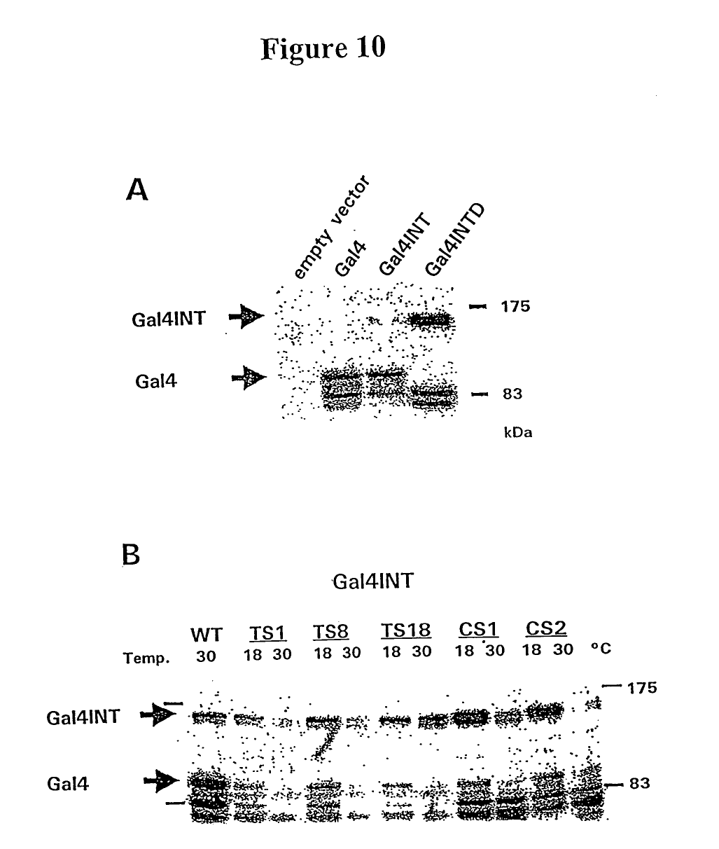

40. The nucleic acid of claim 39, wherein the GAL4:Intein hybrid polypeptide has the sequence shown in FIG. 9.

41. A cell transfected with the nucleic of claim 36.

42. A method for producing a regulatable chimeric polypeptide comprising expressing the nucleic acid of claim 36 in a cell.

43. An assay for identifying an intein self-excision agonist or antagonist compound using a chimeric polypeptide comprising a target polypeptide which encodes a bioactivity and an intein polypeptide inserted into the target polypeptide comprising:
contacting the regulatable chimeric polypeptide with a test compound; and
measuring the bioactivity of the target polypeptide
wherein a statistically significant increase in the target polypeptide bioactivity in the presence of the test compound, in comparison to the target polypeptide bioactivity in the absence of the test compound, indicates that the test compound is an intein self-excision agonist compound while a statistically significant decrease in the target polypeptide bioactivity in the presence of the test compound, in comparison to the target polypeptide bioactivity in the absence of the test compound, indicates that the test compound is an intein self-excision antagonist compound.

44. A nucleic acid cloning vector for use in creating a regulatable chimeric polypeptide from a target polypeptide-encoding nucleic acid sequence comprising:
a cloning site for an N-Extein-encoding nucleic acid sequence;
a regulatable intein-encoding sequence; and
a cloning site for a C-Extein-encoding nucleic acid sequence
wherein the N-Extein-encoding nucleic acid sequence to be inserted encodes an amino-terminal portion of the target polypeptide and the C-Extein-encoding nucleic acid to be inserted encodes a carboxy-terminal portion of the target polpeptide.

45. The nucleic of claim 44, which further comprises a transcriptional regulatory sequence.

46. A kit comprising the cloning vector of claim 44.

47. The kit of claim 46, further comprising a compound which is an agonist or antagonist of the regulatable intein encoded by the regulatable intein-encoding sequence of the cloning vector.

48. The kit of claim 46, further comprising at least one additional cloning vector in which the reading frame between the N-Extein cloning site and the regulatable intein-encoding sequence or between the regulatable intein and the C-Extein cloning site has been changed by the addition of one or two nucleotides or some multiple of one or two nucleotides.

49. A method of regulating the level of a target polypeptide comprising:
providing a target polypeptide containing at least one internal cysteine residue;
inserting a conditional intein with a self-excision activity into said target polypeptide upstream of the internal cysteine residue to produce an unspliced target-intein precursor protein; and
providing a signal that agonizes or antagonizes the intein self-excision activity, thereby
increasing or decreasing the level of the mature spliced target polypeptide.

50. The method of claim 49, wherein the target polypeptide is selected from the group consisting of: Gal4, Gal80 and GFP.
